SEQUENCE LISTING (25 sequences)

SEQ ID NO: 1 - protein, 19 aa, UNKNOWN
  Synthesized
LLNFDLLKLAGDVESNPGP

SEQ ID NO: 2 - protein, 33 aa, UNKNOWN
  Synthesized
EARHKQKIVAPVKQTLNFDLLKLAGDVESNPGP

SEQ ID NO: 3 - protein, 30 aa, UNKNOWN
  Synthesized
HKQKIVAPVKQTLNFDLLKLAGDVESNPGP

SEQ ID NO: 4 - protein, 7 aa, UNKNOWN
  synthesized
DXEXNPG

SEQ ID NO: 5 - protein, 18 aa, UNKNOWN
  Synthesized
QGWVPDLTVDGDVESNPG

SEQ ID NO: 6 - protein, 18 aa, UNKNOWN
  Synthesized
GGGQKDLTQDGDIESNPG

SEQ ID NO: 7 - protein, 10 aa, UNKNOWN
  Synthesized
PFLSGLLGTV

SEQ ID NO: 8 - protein, 4 aa, UNKNOWN
  Synthesized
VTAQ

SEQ ID NO: 9 - protein, 4 aa, UNKNOWN
  Synthesized
VTAM

SEQ ID NO: 10 - protein, 4 aa, UNKNOWN
  Synthesized
VTAN

SEQ ID NO: 11 - protein, 13 aa, UNKNOWN
  synthesized
DLTQDGDIEXNPG

SEQ ID NO: 12 - protein, 20 aa, UNKNOWN
  synthesized
IIGGGQKDLTQDGDIEXNPG

SEQ ID NO: 13 - protein, 20 aa, UNKNOWN
  synthesized
NIIGGGQKDLTQDGIEXNPG

SEQ ID NO: 14 - protein, 24 aa, UNKNOWN
  synthesized
RRQNIIGGGQKDLTQDGDIEXNPG

SEQ ID NO: 15 - protein, 15 aa, UNKNOWN
  Synthesized
KVNFVQTTPVVVAAK

SEQ ID NO: 16 - protein, 4 aa, UNKNOWN
  Synthesized
VAAK

SEQ ID NO: 17 - protein, 12 aa, UNKNOWN
  Synthesized
FVQTTPVVVAAK

SEQ ID NO: 18 - protein, 4 aa, UNKNOWN
  Synthesized
VEAQ

SEQ ID NO: 19 - protein, 20 aa, UNKNOWN
  Synthesized
FILKNVMLAVGAVMLAYKVY

SEQ ID NO: 20 - protein, 20 aa, UNKNOWN
  Synthesized
QLLNFDLLKLAGDVESNPGP

SEQ ID NO: 21 - protein, 14 aa, UNKNOWN
  Synthesized
MMINPQQPIQVTAQ

SEQ ID NO: 22 - protein, 254 aa, UNKNOWN
  Synthesized
GDEDTPAGELSIEQDTHKNTTLAESTDASTAYVATEEFSMMPWITDGPHVYPDLTERWTKAFQFQWTTSQAQGEIIQRFDLPIEAIQNFINSPNALPWRQHAFYKSDIELKVQVNSQPGQSGYLILGAMYEASEGTAIGNRVDHAANIVAMPHMRISAGASNSGDMVIPYIRHFPVGCILNNAFDVPQYFVTLFVAPLLQLRTGADGPQVVDVTIMIRFPNCEFYGNRTTENIVTANGWVPDLTVDGDVESNPG

SEQ ID NO: 23 - protein, 333 aa, UNKNOWN
  Synthesized
PFLSGLLGTVAAIGKTVASAGSSGSIASGVSGVANGIGSFGSAAGKVIGGVESLLRPLFPKKDMDRPQNIIEPTNFYLQQNTSLSLATGTNNVKLLQLQAENSVSHPPGFVPVDDQFNNRFITSVFGKSDYFQWTSDAASGTLLYSFDVTPLKSFVRGIGTQPTSYLTPMAALAGNYGGYHGDLEMRLTFAVSKFHSGRIFIVYSPDVVPTFDNIGAYYSVLLDVQDQSVYTFKIPYNAPTPYVPLFEALNGDNGVFVLPPGGPSINAVCMAYGYVSIFVENQLRVMQTAAPTIDVLAELRGADNFHLVLPAGGKFRSIATTATAEASVVTAM

SEQ ID NO: 24 - protein, 284 aa, UNKNOWN
  synthesized
GDERREFHTVNPTPRTIIPVWVAQLNESYDCRDVVKRYHDWFDIVSPAIVAGRGFPNIYVNVTVFHVPVPAFDAVPIQRVQFTDISIENSMLNKSMVLARERINLATNNTISIRVPWTNYACMISNSINPASGRVNTMAPYSNGRVVVYVEYLSPYTLTQGVFKVVWDVTAGSASTRPDTFTPDMLTLLHDGFRFAKGDFNYQLDFTPAPCGSNTAIRLRCYRSYGDGGNLYVFQGFPRMIGNYVPRQAISGITPTRGGLRRQNIIGGGQKDLTQDGDIEXNPG

SEQ ID NO: 25 - protein, 41 aa, UNKNOWN
  Synthesized
PTQSVEAQARDYDQKTEAPNTPKVNFVQTTPVVVAAKGDEG

(19) United States
(12) Patent Application Publication
(10) Pub. No.: US 2011/0136730 A1
(43) Pub. Date: Jun. 9, 2011

(54) "Method for screening compounds comprising the use of picornavirus protease 2A"

(76) Inventors: John Fuller Atkins, Salt Lake City, UT (US); Martin Ryan, Newton-on-Tay (GB); Gary Loughran, County Cork (IE)

(21) Appl. No.: 12/736,552

(22) PCT Filed: Apr. 17, 2009
(86) PCT No.: PCT/IE2009/000019

§ 371 Date: Dec. 14, 2010

Related U.S. Application Data
(60) Provisional application No. 61/071,272, filed on Apr. 18, 2008.

(51) Int. Cl. A61K 38/12; C12Q 1/68; C07H 21/00; C07K 11/02; A61P 31/14

(52) U.S. Cl. 514/3.7; 435/6.1; 536/23.1; 530/317

(57) ABSTRACT

A method of screening compounds or molecules comprising the steps of: translating a sequence encoding the amino acid sequence comprising SEQ ID No. 29 in a translation system in the presence of a test compound or molecule; and analysing the translation product(s) for the presence of one or more of (a) a peptide comprising the amino acids Pro-Gly at the C terminus and a peptide comprising the amino acid Pro at the N terminus: or (b) a peptide comprising the amino acid sequence of SEQ ID No. 29.

This invention relates to a method of screening compounds.

Protein synthesis (translation) takes place in three phases: initiation, elongation and termination. All of these phases involve ribosomes. Ribosomes contain two subunits each of which contains numerous proteins and RNA. Recognition by the anticodon of transfer RNA (tRNA) by messenger RNA (mRNA) codons, which are non-overlapping mRNA base triplets that specify an amino acid, occurs near the interface of the smaller subunit at a site known as the A-site. Transfer RNAs (tRNAs) are somewhat variable in size but the nucleotide position of the anticodon is conventionally called 34-36. The corresponding amino acid is generally acylated to the 3′ end of tRNA (conventionally called nucleotide 76), prior to its entry to the ribosome (Review [Weinger and Strobel, 2006]). During the elongation phase of protein synthesis, peptidyl-tRNA is in a ribosomal site known as the P-site as aminoacyl tRNA enters an adjacent site known as the A-site. Extension of the growing polypeptide chain involves reaction of the α-amino group of aminoacyl-tRNA bound to the A-site with the ester carbon of peptidyl-tRNA bound in the P-site (Review [Green and Lorsch, 2002]) and is facilitated by ribosomal RNA (rRNA) undergoing movements that reorient the ester linkage making it accessible for this attack [Schmeing et al, 2005 a and b]. This occurs at the peptidyl-transfer centre (PTC) on the interface of the larger subunit. The A-site tRNA is moved to the peptidyl-tRNA, or P-site, of the ribosome and subsequently, after it is deacylated, tRNA enters the ribosomal E-site and then exits the ribosome. In standard decoding, the PTC positions the ester linkage so that it is not accessible to nucleophilic attack by water with resulting deacylation of peptidyl-tRNA [Schmeing et al, 2005a]. In contrast, at the end of a coding sequence when a termination codon (UAG, UAA or UGA in nearly but not quite all, cases) enters the A-site, release is not mediated by tRNAs but rather as a consequence of conformational changes following the interaction of specific ‘release factor’ proteins with the ribosome.

The path of the nascent peptide chain from the internal site of peptide bond formation through the body of the larger ribosomal subunit to the outside surface of the larger subunit is known as the peptide exit tunnel (it may be considered as the birth canal of the ribosome). The peptide exit tunnel extends through the larger ribosomal subunit from the peptidyl transferase site on the subunit interface to the site where the peptide exits [Milligan and Unwin, 1986; Yonath et al, 1987]. Whilst there is little structural information on the exit tunnel of eukaryotic ribosomes, the structure of the prokaryotic ribosome is well documented, for example, the exit tunnel in the archea, Haloarcula marismortui, is an unbranched “tube” which has been reported to have dimensions that may accommodate alpha-helices but it is not wide enough to accommodate polypeptides with more elaborate structure than alpha-helices [Voss et al, 2006]. Consistent with the interpretation of early work, Malkin and Rich (1967) and Blobel and Sabatini (1970) have shown that 30 to 40 amino acids are protected by the ribosome, they have also calculated the dimensions of the exit tunnel. The walls of the tunnel in bacteria and archaea are comprised predominantly of rRNA and the nonglobular regions of three proteins. Two of these proteins, which are designated L4 and L22 in bacteria, are situated near the entrance to the tunnel, and the third protein, L23, (bacterial nomenclature) resides near the exit site. In bacteria pronounced constriction in the tunnel is located about 30 Å from the peptidyl-transferase centre at a point where a conserved 13 hairpin loop of L22 comes into close proximity to L4.

Certain nascent peptide chains, while still within the ribosome, can influence translation of downstream sequences in the same mRNA that encoded them. These nascent chains can be encoded by short open reading frames (ORFs) and influence initiation of translation of the main and 3′ ORF in the same RNA. Alternatively these nascent peptides can influence continuing downstream translation of the same coding sequence that encoded them. Synthesis of E. coli secretory monitor SecM involves a nascent peptide effect and a study of mutants which affected its synthesis provided direct evidence for sensing by the exit tunnel [Nakatogawa and Ito, 2002], this study also led to the concept of a discriminating gate in the exit tunnel. Subsequent studies using fluorescence transfer [Woolhead et al, 2006] and cryo Electron Microscopy [Mitra et al, 2006] provided evidence for a series of reciprocal interactions between the ribosome and the C terminus of the nascent SecM polypeptide. In some cases the effect of nascent peptide sequences within the ribosome is modulated by specific small molecules. For instance tryptophan influences completion of decoding a leader ORF in a bacterial mRNA that encodes tryptophanase [Cruz-Vera et al, 2006], arginine affects translation of a 5′ leader of fungal carbamoyl phosphate synthetase mRNA [Wang et al, 1998] and spermidine modulates translation of a leader of mammalian S-adenosylmethionine decarboxylase mRNAs [Raney et al, 2002].

The foot-and-mouth disease virus is a member of the Picornaviridae family. Picornaviruses are non-enveloped positive stranded RNA viruses with an icosahedral capsid. Foot-and-mouth disease virus is probably the most contagious mammalian virus. It has a wide host-range and causes disease in a number of domesticated animal species (cattle, sheep, pigs and goats). Although only rarely fatal, the virus causes long-term reduction (˜20%) in productivity of animals which have recovered from the virus. The virus may also establish persistent infections producing ‘carrier’ animals which may act as a source of infection for naive animals. There is currently no treatment for infected livestock. The main disease control measure is mass slaughter (potentially millions of animals) resulting in large economic losses. Vaccination of healthy animals is possible however, there are seven distinct serotypes of the foot-and-mouth disease virus (O, A, C, South African Territories (SAT) 1, SAT2, SAT3, and Asia 1) each of which stimulates a different immune response. Immunity to one serotype does not protect against infection caused by a different serotype, the commercially available vaccines are specific for each serotype. Furthermore, current vaccines are expensive and the immunity conferred by vaccination tends to be short-lived (about 6 months protection at best) so in those countries that do vaccinate, livestock must be frequently revaccinated at great expense. There is also the danger that animals vaccinated against one serotype may become carriers if exposed to new infection of the virus. In those countries designated as foot-and-mouth disease free without vaccination (such as the US and the UK) all the susceptible animals within a certain radius of an outbreak of foot-and-mouth disease are slaughtered. Another, equally significant, economic loss caused by an outbreak of foot-and-mouth disease is that incurred by the tourism industry since restrictions to the access of the countryside impact upon many rural leisure pursuits.

During infection, once the RNA has been injected into a host cell, the RNA is replicated and translated by ribosomes of the host cell.

### STATEMENTS OF INVENTION

According to the invention there is provided a method of screening compounds or molecules comprising the steps of:
translating a sequence encoding the amino acid sequence comprising SEQ ID No. 29 in a translation system in the presence of a test compound or molecule; and
analysing the translation product(s) for the presence of one or more of
   a) a peptide comprising the amino acids Pro-Gly at the C terminus and a peptide comprising the amino acid Pro at the N terminus; or
   b) a peptide comprising the amino acid sequence of SEQ ID No. 29.

In another aspect, we describe a method for identifying a compound and/or molecule suitable for modifying protein synthesis comprising the steps of:
translating a sequence encoding the amino acid sequence comprising SEQ ID No. 29 in a translation system in the presence of a test compound and/or molecule; and
analysing the translation product(s) produced
   wherein a compound and/or molecule suitable for modifying protein synthesis is identified by the presence of the amino acid sequence of SEQ ID No. 29 in the translation product(s).

The presence of a peptide comprising the amino acid sequence of SEQ ID No. 29 in the translation product(s) is indicative of the ability of the test compound or molecule to disrupt and/or prevent a translational recoding event (StopGo).

The sequence may encode the amino acid sequence comprising SEQ ID No. 30. The amino acid in position 1 may be selected from asparagine, histidine, glutamate, glutamine and lysine. The amino acid position 1 may be asparagine. The sequence may encode the amino acid sequence comprising SEQ ID No. 35. The amino acid in position 1 may be selected from aspartate and asparagine. The amino acid in position 2 may be selected from valine and isoleucine. The amino acid in position 3 may be selected from glutamate, aspartate and glycine. The amino acid in position 4 may be selected from serine, isoleucine, phenylalanine, threonine, alanine, glutamate, and methionine. The amino acid in position 5 may be selected from asparagine, histidine, glutamate, glutamine and lysine.

The sequence may encode the amino acid sequence comprising SEQ ID No. 31.

The sequence may encode the amino acid sequence comprising SEQ ID No. 1.

The sequence may encode the amino acid sequence comprising SEQ ID No. 2.

The method may comprise the step of cloning a sequence encoding the amino acid sequence comprising SEQ ID No. 29 into a plasmid prior to the step of translating the sequence.

The sequence may be translated in a cell free system, such as a rabbit reticulate lysate system. The translation system may be a coupled or uncoupled transcription/translation system. Alternatively, the sequence may be translated in a cell based system.

The sequence may comprise a detection tag. The detection tag may be located downstream of the sequence encoding the C terminal proline residue of the amino acid sequence of SEQ ID No. 29. The sequence may comprise a second detection tag. The second detection tag may be located upstream of the sequence encoding the N terminal proline residue of the amino acid sequence of SEQ ID No. 29.

The detection tag may be a radiolabelled amino acid, such as ³⁵S methionine. The translation product(s) may be analysed using gel electrophoresis. The translation product(s) may be analysed using phosphorimaging. The detection tag may be luminescent. The detection tag may be fluorescent. The detection tag may be a fluorescent fusion protein. The fluorescent protein may be selected from: green fluorescent protein, yellow fluorescent protein, cyan fluorescent protein, cherry red fluorescent protein, and tomato red fluorescent protein.

The translation product(s) may be analysed using gel electrophoresis. The translation product(s) may be analysed using luminescence detection. The translation product(s) may be analysed using fluorescent imaging.

The first and second detection tags may be different. The translation product(s) may be analysed using fluorescent resonance energy transfer analysis.

The detection tag may be an enzyme. The detection tag may be a luciferase enzyme. The detection tag may be selected from firefly luciferase and renilla luciferase. The first and second detection tags may be different. The first detection tag may be firefly luciferase or renilla luciferase. The second detection tag may be renilla luciferase or firefly luciferase.

The translation product(s) may be analysed by the steps of:
adding a substrate for the first detection tag;
quantifying the luminescent signal produced by the first detection tag;
adding a quencher to quench the first detection tag reaction;
adding a substrate for the second detection tag; and
quantifying the luminescent signal produced by the second detection tag.

The steps of adding a quencher to quench the first detection tag reaction and adding a substrate for the second detection tag may be performed simultaneously.

The translation product(s) may be analysed by the steps of:
dividing the translation system into two sub populations;
adding a substrate for the first detection tag to a first sub population;
quantifying the luminescent signal produced by the first detection tag;
adding a substrate for the second detection tag to a second sub population; and
quantifying the luminescent signal produced by the second detection tag.

The translation system may comprise cells. The cells may be broken prior to being divided into two sub populations. The substrate for the first detection tag and the substrate for the second detection tag may be added simultaneously.

The substrate for the first detection tag may be coelenterazine. The substrate for the second detection tag may be luciferin.

The translation product(s) may be analysed by Western Blotting.

The invention also provides the use of a sequence encoding the amino acid sequence comprising SEQ ID No. 29 for identifying a compound and/or a molecule for the prophylaxis and/or treatment of a virus.

The sequence may encode the amino acid sequence comprising SEQ ID No. 30. The amino acid in position 1 may be selected from asparagine, histidine, glutamate, glutamine and lysine. The amino acid in position may be asparagine.

The sequence may encode the amino acid sequence comprising SEQ ID No. 35. The amino acid in position 1 may be selected from aspartate and apsargine. The amino acid in position 2 may be selected from valine and isoleucine. The amino acid in position 3 may be selected from glutamate, aspartate and glycine. The amino acid in position 4 may be selected from serine, isoleucine, phenylalanine, threonine, alanine, glutamate, and methionine. The amino acid in position 5 may be selected from asparagine, histidine, glutamate, glutamine and lysine. The sequence may encode the amino acid sequence comprising SEQ ID No. 31.

The sequence may encode the amino acid sequence comprising SEQ ID No. 1.

The sequence may encode the amino acid sequence comprising SEQ ID No. 2. The virus may be selected from one or more of: Foot and Mouth disease virus, Vilyuisk virus, Saffold virus, encephalomycarditis virus, Theiler's murine encephalitis virus and Parecho-type viruses.

The invention further provides a compound and/or molecule identified by the method as described herein. The invention also provides for the use of a compound and/or molecule in the prophylaxis and/or treatment of a viral disease such as Foot and Mouth disease. The invention further provides a method of treatment and/or prophylaxis of a viral disease such as Foot and Mouth disease comprising the step of administering an effective amount of a compound and/or a molecule to a mammal.

The invention also provides a kit comprising a translation system and a sequence encoding the amino acid sequence comprising SEQ ID No. 29. The kit may comprise a means for analysing a translation product(s). The translation system may be any one or more of the translation systems described herein. The means for analysing a translation product may comprise any one or more of the analysis means described herein. The kit may further comprise instructions for use and/or instructions for analysing a translation product(s).

We also describe an in vivo (tissue culture) and in vitro method for identifying compounds and/or molecules suitable for modifying protein synthesis comprising the steps of:
creating plasmids encoding a StopGo protein flanked by two luciferase or fluorescent reporter proteins (or a combination of both); and
translating the plasmid in a cell and in a cell-free translation system in the presence and/or absence of compounds and/or molecules to be tested; and
analysing the translation products produced
   wherein compounds and/or molecules suitable for inhibiting StopGo are identified by the presence of an at least four-fold increase in the ratio of firefly/renilla luciferase when compared to non-inhibition of StopGo and the presence of a full length translation product as assessed by Western blots probed with antibodies raised against either of the luciferase or fluorescent proteins.

The plasmid may be a cDNA plasmid and may further comprise a fluorescent protein for example a green fluorescent protein encoding sequence. The plasmid may further comprise firefly luciferase encoding sequences or derivatives thereof. The plasmid may further comprise renilla luciferase encoding sequences or derivatives thereof. The plasmid may further comprise β-glucuronidase encoding sequences. The plasmid may have an internal ribosomal entry site (IRES). The plasmid may encode SEQ ID NO. 20. The translation system may be a coupled or uncoupled transcription/translation system, for example a rabbit reticulocyte lysate system. The translation system may be a cell-line either stably or transiently transfected with any of the above mentioned plasmids. The translation system may be a cell-free extract derived from tissue culture cell-lines

In one aspect, the method may further comprise the step of incorporating a radio labelled amino acid into the translation system. The radio labelled amino acid may be ³⁵S methionine. The translation products may be analysed using gel electrophoresis. The translation products may be analysed using phosphorimaging. The translation products may be qualified by phosphorimaging.

In a further aspect, the method further comprises the step of incorporating a fluorescent label into the translation system. The translation products may be analysed using gel electrophoresis. The translation products may be analysed using fluorescent imaging. The translation products may be quantified using fluorescent imaging.

We also describe a method for identifying compounds and/or molecules suitable for modifying protein synthesis comprising the steps of:
creating a plasmid comprising a first and second fluorescent proteins and encoding the StopGo protein;
transfecting cells with the plasmid construct;
culturing the transfected cells in the presence and/or absence of compounds and/or molecules to be tested; and
analysing proteins produced by the cultured cells.

The plasmid may further comprise a co-translational signal sequence. For example the co-translational signal sequence may be a GaLT leader sequence and or a Calrectiticilin leader sequence commercially available GaLT and/or calreticulin leader sequences are suitable for use with the invention. The co-translational signal may be located immediately downstream of the sequence encoding StopGo.

The first fluorescent protein may be yellow fluorescent protein. The second fluorescent protein may be cyan fluorescent protein. The cultured cells may be analysed using fluorescence resonance energy transfer (FRET) analysis.

In a further aspect we describe a method for identifying compounds and/or molecules suitable for modifying protein synthesis comprising the steps of:
creating a reporter construct comprising a first and second enzyme and encoding the StopGo protein;
transfecting cells with the reporter construct;
culturing the transfected cells in the presence and/or absence of compounds and/or molecules to be tested; and
analysing proteins produced by the cultured cells
   wherein compounds and/or molecules suitable for modifying protein synthesis are identified by the production of a protein comprising the first and second enzymes.

The sequence encoding StopGo may be SEQ ID No. 3. The first enzyme may be fused to the 5′ end of the sequence encoding SEQ ID NO. 3. The first enzyme may be firefly or Renilla luciferase. The first enzyme and the sequence encoding SEQ ID No. 3 may be fused to the 5′ end of the second enzyme. The second enzyme may be firefly or Renilla luciferase.

The step of analysing proteins produced by the cultured cells may comprise the steps of:
Either splitting the cells into two separate populations and then adding a substrate for firefly into one cell population;
quantifying the luminescent signal produced; and adding a substrate for Renilla into the second cell population; and
quantifying the luminescent signal produced.

Alternatively the cells may be analysed without splitting by:
adding a substrate for firefly and quantifying the luminescent signal produced;
adding a quencher to quench the firefly reaction;
adding a substrate for Renilla; and
quantifying the luminescent signal produced by the second enzyme.

The steps of adding a firefly quencher and adding a Renilla substrate may be performed simultaneously. The substrate for firefly luciferase may be luciferin. The quencher may be Stop and Glo® (Promega). The substrate for Renilla luciferase may be coelenterazine. The proteins produced by the cultured cells may be analysed by Western Blotting.

The compounds and/or molecules identified by the methods described herein may reduce or prevent StopGo translation. The compounds and/or molecules identified by the methods described herein may be used in the prophylaxis and/or treatment of foot and mouth disease.

We also describe a method of treatment and/or prophylaxis of foot and mouth disease comprising the step of administering an effective amount of a compound and/or molecules identified by the methods described herein to a mammal.

### BRIEF DESCRIPTION OF THE DRAWINGS

The invention will be more clearly understood from the following description of an embodiment thereof, given by way of example only, with reference to the accompanying drawings, in which:—

FIG. 1 is a schematic diagram of the protein domains found in the polyprotein Foot-and-Mouth disease. The dotted arrows indicate functional domains of the polyprotein and the solid arrows indicate ‘secondary’ processing of the polyprotein;

FIG. 2 is a schematic representation of StopGo generating two discrete proteins encoded by an uninterrupted open reading frame (ORF). Deacylated tRNA^{Pro} is represented on the left exiting the ribosomal E-site. Peptidyl-tRNA^{Gly} is depicted in the centre in the ribosomal P-site with aminoacyl-tRNA^{Pro} on the right in the ribosomal A-site. The barrier to peptidyl transfer is diagrammed as solid black bar and the importance of the closest part of the nascent chain is symbolized by a bracket and dotted line and partly below by the 16 in parenthesis followed by PRO GLY. The left bar below represents the C-terminal end of the protein encoded upstream, while the right bar below represent the N-terminal end of the downstream encoded protein;

FIG. 3 is a schematic comparison of the genome organisation of Perina nuda picorna-like virus (PnPV) and Foot and Mouth disease (FMDV). The protein products of a single open reading frame are shown as separate boxes. The shaded regions were identified by intact or digested protein analysis of PnPV virions. “L” denotes the leader peptide. Regions encoding 2A sequences are overlined and indicated by arrows. Putative virally encoded protease cleavage sites in PnPV are indicated by “*”. Only one of the virally encoded protease cleavage sites *, is indicated in FMDV. CP1-4 denotes the four PnPV capsid proteins and VP1-4 denotes the four FMDV capsid proteins;

FIG. 4 illustrates analysis of PnPV virion digests by Fourier transform-ion cyclotron resonance (FT-ICR). The single 2986 amino acid open reading frame (ORF) of PnPV is depicted in sections. Underlined sequences were identified in tryptic or chymotryptic digests of virions. The two 2A sequences are italicised and the most crucial residues for StopGo are shown in white with black shading. Putative viral protease recognition sequences are shaded in grey. “pS” indicates phosphorylation of S₁₁₈₇. The bold “Y” is the residue expected to link the putative VPg to the 5′ end of PnPV RNA;

FIG. 5 is an electrospray molecular-mass spectra. (A) is the mass spectrum of denatured virions showing the four major coat proteins, CP1-4. (B) are expanded views of the regions around the major peaks for CP1-4. Potassium (K) adducts and oxidation products are indicated. Oxidation of cysteine, proline, methionine, or other residues in the protein may have occurred from storage of the virus sample, exposure to reagents, reaction with degraded β-mercaptoethanol in solution, or may be due to post-translational modifications;

FIG. 6 is a schematic representation indicating the location of an oligopeptide sequence to which the activity of 2A and ‘2A-like’ sequence of foot-and-mouth disease virus have been mapped;

FIG. 7 (A) is a schematic of the wild type (WT) and mutant (Mu) translation products; (B) is a bar chart showing the relative luciferase activity of the wild type (WT) and mutant (Mu) translation products; and (C) is a Western Blot of the wild type (WT) and mutant (Mu) constructs of the dual firefly reporter system of Example 4 the top panel shows firefly as the α reporter (as illustrated in (A)) and the bottom panel shows Renilla as the α reporter;

FIG. 8 is a sequence alignment of putative StopGo sequences identified by a homology search of protein databases;

FIG. 9 is a Western Blot of putative bovine and human StopGo sequences using the dual luciferase assay in accordance with Examples 4 and 5 the top panel shows the firefly as the α reporter in the dual luciferase system and the bottom panel shows Renilla as the α reporter in the dual luciferase system; and

FIG. 10 is a bar chart showing the effect of known ribosomal targeting antibiotics on StopGo activity.

### DETAILED DESCRIPTION

In 1982, two years before the completed nucleotide sequence of encephalomyocarditis virus (EMCV) was elucidated, Anne Palmenberg, [Palmenberg and Rueckert, 1982] reported an unusual intramolecular self-cleavage of an EMCV protein. She noted that this cleavage activity was not sensitive to dilution which suggested that a typical enzymatic protease was not involved. A few years later, Richard Jackson [1986] noted that the EMCV cleavage activity, which occurred while the protein was still nascent in the ribosome, was the primary cleavage event and was resistant to high temperatures and high concentrations of protease inhibitors. In 1989, Martin Ryan [Ryan et al., 1989] showed that in Foot and Mouth disease virus (FMDV) the cleavage between 2A and 2B proteins was independent of the viral-encoded proteases and was still shown to occur when 2A and as few as four 2B N-terminal amino acids were present. A couple of years later Batson and Rundell [1991] demonstrated similar results for Theiler's murine encephalitis virus (TMEV).

It was previously known [Robertson et al. 1985] that FMDV 2A protein was small. Originally, the FMDV 2A protein was believed to be only 16 amino acids in length but it was later shown to be 18 amino acids long. The FMDV 2A protein shared some amino acid homology with the C-terminus of the EMCV and TMEV 2A proteins. Although EMCV and TMEV 2A proteins are about 150 amino acids long they share no amino acid homology with 2A proteins from the closely related entero- and rhinoviruses.

In 1992, Anne Palmenberg's work on EMCV showed that insertional mutagenesis of a conserved 2A C-terminal amino acid sequence NPGP (SEQ ID No. 30) could completely disrupt the cleavage of the EMCV 2A protein [Palmenberg et al., 1992]. Using point mutation analysis Palmenberg demonstrated that PGP in particular were essential amino acids for cleavage of the 2A protein and that this cleavage event was a requirement for propagation of the virus [Hahn and Palmenberg, 1996].

Through a series of deletion mutagenesis studies [Ryan et al. 1994] it was revealed that as little as twelve 2A amino acids plus the N-terminal proline of 2B were sufficient to allow cleavage to proceed. A few years later, Donnelly et al [1997] showed that eighteen 2A amino acids from both TMEV and EMCV were similarly sufficient to direct cleavage even though their 2A proteins were about 150 amino acids in length. It wasn't until 2001 that Donnelly et al proposed the first mechanism for the 2A/2B cleavage observed in FMDV, TMEV and EMCV [Donnelly et al., 2001b] suggesting that there was a ribosomal ‘skip’ from one codon to the next without the formation of a peptide bond. In addition they showed, that the amino acid requirement for this event extended beyond the highly conserved DXEXNPGP (SEQ ID No. 28) 2A amino acid sequence strongly suggesting that the preceding (upstream) sequences are having some effect within the exit tunnel of the ribosome [Donnelly et al 2001a]. In the same year, the identification of active 2A/2B cleavages in other more disparate virus families were described. Up to this point, scientists had been focussing on the use of the 19 amino acid sequence to direct multiple protein synthesis (cleaved and uncleaved proteins) from a single sequence as a tool for biotechnology protein production.

In contrast to protein changes redefinition of an mRNA codon meaning results in a competition between the standard meaning of the codon (such as coding an amino acid) and the redefined meaning (such as termination of translation). During the synthesis of the FMDV 2A protein, we have shown that a glycine codon within a 2A encoding sequence specifies the expected amino acid but also promotes subsequent termination of translation of that chain with continued translation resulting in the next encoded amino acid becoming the N-terminal residue of a separate downstream encoded polypeptide [Atkins et al, 2007]. Up until this point, the term “2A cleavage” was used but we proposed the term “StopGo” as there is no evidence that the C terminus of the product encoded upstream and the N terminus of the downstream-encoded product are ever joined by a peptide bond, therefore StopGo is not a cleavage mechanism but a translational recoding event.

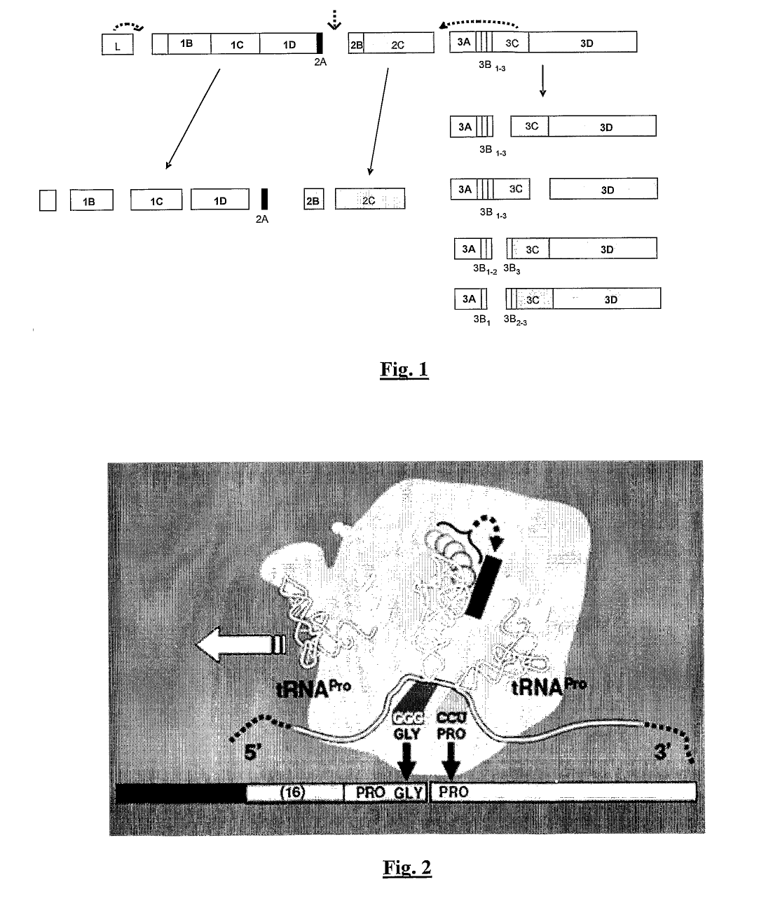

Picornavirus proteins are encoded in the form of a single, long, open reading frame (ORF). The full-length translation product (predicted by inspection of the nucleotide sequence) is not observed within infected cells due to both co- and post-translational polyprotein ‘processing’ [Ryan and Flint, 1997]. Co-translational ‘primary’ polyprotein cleavages serve to separate the polyprotein into functional domains (dotted arrows of FIG. 1). In the case of the aphthovirus Foot-and-Mouth disease virus (FMDV) the RNA strand has an IRES mediated translation start site followed by a single long ORF from which multiple products are derived. The polyprotein comprises the L proteinase (L^{pro}), capsid proteins 1A, 1B, 1C and 1D (P1), proteins 2A, 2B, 2C, 3A, 3B1, 3B2, 3B3 (3B₁₋₃), the 3C proteinase (3C^{pro}) and the 3D RNA-dependent polymerase (3D^{pol}). The first primary polyprotein processing event is mediated by L^{pro} cleaving at it's own C-terminus (between L^{pro} and capsid protein 1A). The second primary ‘cleavage’ occurs at the C-terminus of 2A (between proteins 2A and 2B), thereby liberating [P1-2A]. The third primary cleavage (between proteins 2C and 3A) is mediated by 3C^{pro}, liberating [3A-3B₁₋₃-3C-3D] or (P3). Subsequent, ‘secondary’ polyprotein processing is mediated by the 3C^{pro} (solid arrows of FIG. 1).

In Foot and Mouth disease virus (FMDV) the 2A region of the polyprotein is only 18 amino acids long. The boundaries of this region were defined by N-terminal sequencing of the (downstream) protein 2B [Robertson et al, 1985]. It has been demonstrated that 2A is proteolytically ‘trimmed’ away from the upstream capsid protein (1D) by the 3C proteinase [Ryan et al, 1989] at a 3C^{pro} scissile amino acid pair conserved amongst different FMDV strains: a ‘secondary’, post-translational, processing event. Analyses of the polyprotein processing of deletion forms of the FMDV polyprotein indicated that the 2A region could function as an autonomous element, and did not act as a substrate for neither the FMDV proteinases L nor 3C [Ryan et al, 1991a]. Insertion of the sequence encoding 2A sequence, plus the N-terminal residue (proline) of protein 2B into an artificial polyprotein synthesis system demonstrated that 2A, (plus the N-terminal proline of 2B) was sufficient to mediate ‘cleavage’ at the C-tei minus of 2A [Ryan and Drew, 1994]. This N-terminal proline of 2B was later shown to be absolutely required for ‘cleavage’ activity [Ryan et al 1999b; Donnelly et al, 2001a]. Generation of the N-terminal proline product is cotranslational [Ryan and Drew, 1994] and does not occur when the 2A sequence plus the N terminal proline residue is expressed in prokaryotes [Donnelly et al, 1997]. For simplicity, hereafter the functional entity ‘2A’ will be taken to mean the 2A region plus the N-terminal proline of 2B.

Further analyses of the activity of 2A showed this oligopeptide sequence did not function as a novel type of proteolytic element. Rather, this sequence was shown to mediate a novel translational ‘recoding’ event [Ryan et al 1999b; Donnelly et al, 2001b; deFelipe et al, 2003]. This effect has been termed ‘ribosome skipping’ [Donnelly et al, 2001b] and ‘StopGo’ translation [Atkins et al, 2007]. The mechanism of this form of polyprotein processing is entirely different from proteolysis—a peptide bond is not cleaved at all—but the products produced by this form of translational recoding have the same qualitative appearance as a proteolytic cleavage. FIG. 2 schematically illustrates the StopGo mechanism of translation of a foot and mouth disease polypeptide. In the literature 2A-mediated polyprotein processing is, however, often referred-to as ‘cleavage’ (initially in quotation marks, thereafter often not).

The 3′ ends of Aphthovirus (e.g., FMDV) and Cardiovirus sequences that specify 2A have a consensus sequence encoding the following amino acids: D-X-E-X-N-P-G (SEQ ID No. 4), and is immediately followed by the proline codon that encodes the first amino acid of 2B [Hahn and Palmenberg 1996; Donnelly et al. 1997]. Introduction of point mutants affecting amino acid identities showed its importance for 2A activity [Donnelly et al. 2001a; Ryan et al. 2002]. (2A action stands in contrast to situations where specific peptides traversing the exit tunnel cause inhibition of release factor-dependent peptidyl-tRNA hydrolysis in the peptidyl-transferase center [Cao and Geballe 1996; Cruz-Vera et al. 2006].)

Single open reading frames (ORFs) have been created comprising two reporter genes flanking 2A-encoding sequences. When such constructs are analyzed using in vitro translation systems, termination before the N-terminal proline of the downstream reporter results in a molar excess of the N-terminal reporter compared to the C-terminal reporter, further suggesting that a nonproteolytic mechanism is involved [Donnelly et al. 2001b]. SDS-PAGE analyses of products produced by 2A-mediated translation are consistent with the sizes expected for products, and in particular, with each upstream encoded product having its C-terminal amino acid as glycine corresponding to the last residue of the 2A consensus sequence of SEQ ID No. 4. However, size estimations using SDS-PAGE are not nearly sensitive enough to deduce the identity of the C-terminal amino acid of 2A. It was important to confirm whether or not this C-terminal amino acid is glycine for assessing the StopGo model.

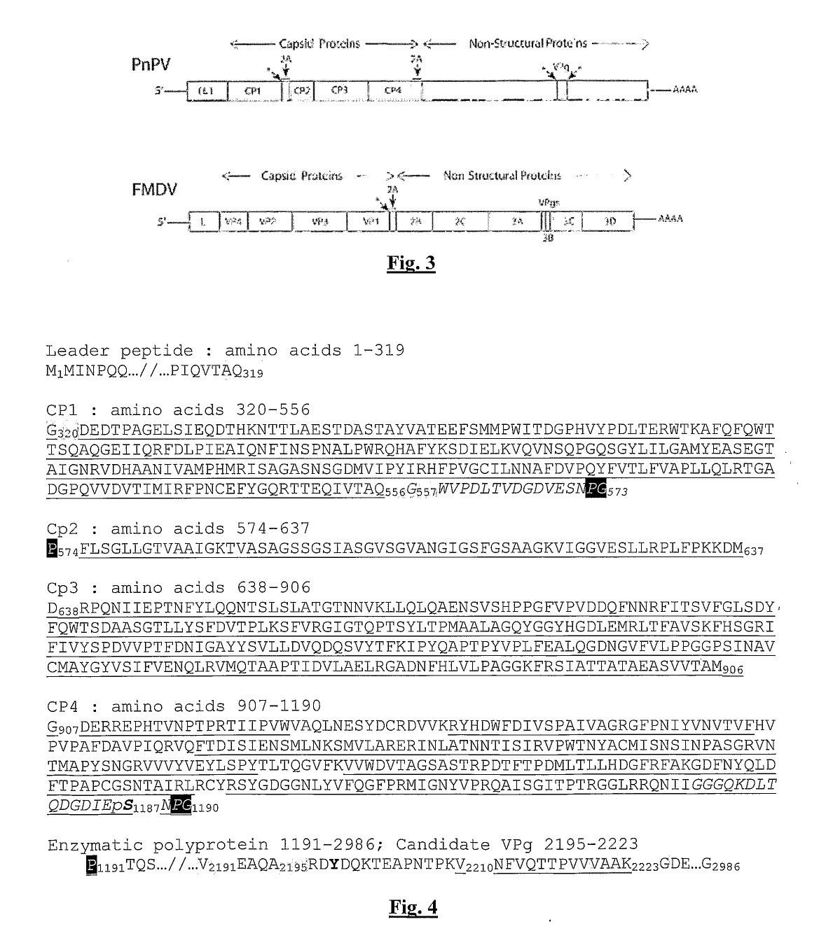

2A-encoding sequences occur in some diverse genes, although nearly all known active 2A-like sequences are from viruses (some occur in non-LTR retrotransposons found in the genomes of a range of species of the parasitic protozoan Trypanosomai) [Heras et al. 2006]. We identified a 2A-encoding viral sequence where the upstream- and the downstream-encoded products are present in the virion and, in theory, readily available from a natural source for accurate sequence characterization by mass spectrometry. The single-stranded RNA Perina nuda picorna-like virus, PnPV, which infects a moth, has these features and also has an additional 2A-encoding sequence where only the upstream-encoded product is present in the virion (FIG. 3; Wu et al. 2002). This virus can be readily propagated in a homologous insect cell line [Wu et al. 2002].

The PnPV genome has a single ORF that has 2986 codons and does not generate subgenomic RNAs [Wu et al. 2002]. It is 89% identical at the amino acid level to Ectropis obliqua picorna-like virus [Wang et al. 2004; Lu et al. 2006], and less so (25%) to Infectious flacherie virus [Isawa et al. 1998]. From the 5′ end of the PnPV ORF there are 319 codons in the position corresponding to the 202 codons in FMDV that encode its leader protease. In both viruses this is just 5′ of the sequence specifying the four capsid coat proteins, which in PnPV are termed CP 1-4. The enzymatic functions, including the polymerase, are encoded 3′ of CP4 (FIG. 3). The first PnPV 2A-encoding RNA sequence, codons 556-573, with the potential to code for QGWVPDLTVDGDVESNPG (SEQ ID NO. 5) is at the 3′ end of the cp1 gene and the first codon, 574, of the adjacent cp2 gene specifies proline. This proline was confirmed by N-terminal sequence analysis [Wu et al. 2002]. The second PnPV 2A-encoding RNA sequence, codons 1173-1190, with the potential to code for GGGQKDLTQDGDIESNPG (SEQ ID NO. 6) is in the vicinity of the predicted 3′ end of the cp4 gene and the 3′ adjacent codon, 1191, is a praline codon [Wu et al. 2002]. N-terminal sequencing of PnPV virion proteins was previously performed by SDS-PAGE fractionation of PnPV virions followed by transfer to PVDF membranes [Wu et al. 2002]. These results defined the beginnings of the four coat protein regions. The N terminus of CP2 is PFLSGLLGTV (SEQ ID No. 7; Wu et al. 2002), consistent with the generation of a new N terminus encoded by the proline codon following the codons for the 2A sequence at the 3′ end of cp1.

PnPV virions, purified as previously described [Wu et al. 2002], were digested with either trypsin or chymotrypsin, and the resulting peptides were analyzed by LC/MS/MS (with Fourier transform-ion cyclotron resonance [FT-ICR]). Complete sequence coverage was obtained for CP2 and CP3, and nearly complete coverage was obtained for CP1 and CP4 (FIG. 4). From the enzymatic digest analyses, all of the N-terminal and C-terminal peptides were identified for the four CP proteins. Of particular interest is the C terminus of CP1 and the amino acid encoded by codon 573. We analysed the peptide masses compared to that predicted from the RNA sequence and identified that the C-terminal amino acid of CP1 is glutamine encoded by codon 556, not glycine as predicted. Peptides containing amino acids encoded by codons 557-573 (GWVPDLTVDGDVESNPG [residues 2 to 18 of SEQ ID NO. 5]) were not detected in the protein digests of isolated capsid components or in the virion sample, as analyzed by LC/MS/MS. The downstream translated protein CP2, however, begins with proline, consistent with previously described products of 2A activity. The peptide coverage for CP2 was complete.

Complete sequence coverage of CP3 was also shown, beginning at codon 638, aspartic acid, and ending at codon 906, methionine. Peptide coverage of CP4 was almost complete except for a few internal peptides (FIG. 4). The N-terminal peptide begins with codon 907, glycine. It was noted that the amino acid sequences at the ends of the leader peptide, CP1 and CP3, ending with VTAQ (SEQ ID NO. 8), and VTAM (SEQ ID NO. 9), respectively, are similar with a consensus sequence of VTAN (SEQ ID NO. 10). In these three cases, the next amino acid encoded is glycine, similar to recognition sequences of picorna viral cysteine proteases that generally cleave between Q (glutamine) or E (glutamate) and G (glycine), A (alanine), or S (serine) [Hellen et al. 1989].

The C-terminal region of CP4 includes the 2A-encoded amino acids and ends at codon 1190, glycine (FIG. 4). In contrast to CP1, the entire 2A-encoded sequence was found to be present at the C terminus of CP4. In addition, analysis of PnPV by LC/MS/MS of trypsin or chymotrypsin digests revealed a post-translational phosphate modification of serine residue 1187. This was confirmed from identification of several different phosphopeptides containing this same serine residue (i.e., from trypsin digestion, DLTQDGDIEpSNPG (SEQ ID NO. 11); from chymotrypsin digestion, IIGGGQKDLTQDGDIEpSNPG (SEQ ID NO. 12), NIIGGGQKDLTQDGDIEpSNPG (SEQ ID NO. 13), and RRQNIIGGGQKDLTQDGDIEpSNPG (SEQ ID NO. 14)). The phosphoserine is near the C-terminal end of CP4 and within the second 2A region. The corresponding nonphosphorylated peptides were also identified, indicating that the phosphorylation is either relatively labile or the extent of phosphorylation is incomplete.

Mass spectrometry analysis of the PnPV virus digests also clearly showed the presence of a peptide encoded 3′ of the CP1-4 region (K)VNFVQTTPVVVAAK- (SEQ ID NO. 15, amino acids 2210-2223). The adjacent downstream amino acid in the polyprotein precursor is glycine. This peptide maps to a region (3B), just N terminal to the putative viral protease (3C) where VPg is found in members of the Picornaviridea (FIG. 3). In Poliovirus and other members of the Picornaviridae, VPg (3B) [Rueckert 1990] is released from the polyprotein by two cleavages at glutamine-glycine amino acid pairs [Nicklin et al. 1986]. It is likely that the C terminus of the PnPV peptide was similarly generated by the action of the viral protease since it ends with VAAK (SEQ ID NO. 16) and is followed by glycine, which is broadly similar to the other PnPV protease cleavage sites (VTAQ (SEQ ID NO. 8)/G in both the putative leader and CP1 and VTAM (SEQ ID NO. 9)/G in CP3). The N terminus of the identified peptide was generated by trypsin cleavage at (K)V. In further support, the peptide (F)VQTTPVVVAAK (SEQ ID NO. 17) was identified from the chymotryptic digest LC/MS/MS data, supporting that this is the C-terminal peptide. Another putative viral protease recognition sequence is encoded by codons 2191-2195, VEAQ (SEQ ID NO. 18)/A, upstream of the detected peptide. Utilization of both protease cleavage sites would generate a 29 amino acid peptide similar to, but larger than, VPgs of studied picornaviruses, that has a critical tyrosine residue near its N terminus. (The tyrosine is involved in the linkage of VPg to the 5′ end of the genomic RNA.) Similar to poliovirus VPg [Reuer et al. 1990], the putative PnPV VPg is preceded by a predicted transmembrane domain, FILKNVMLAVGAVMLAYKVY (SEQ ID NO. 19, amino acids 2162-2181). Therefore we conclude that the peptide identified by mass spectrometry most likely represents the C terminus of the VPg protein of PnPV. The LC/MS/MS data from tryptic or chymotryptic digestion of PnPV was also searched for other possible modifications: uridylylation (on serine, threonine, and tyrosine residues) [Richards et al. 2006] and myristoylation (on lysine and N-terminal glycine residues) [Moscufo et al. 1991] in the entire PnPV sequence, and neither of these modifications was identified.

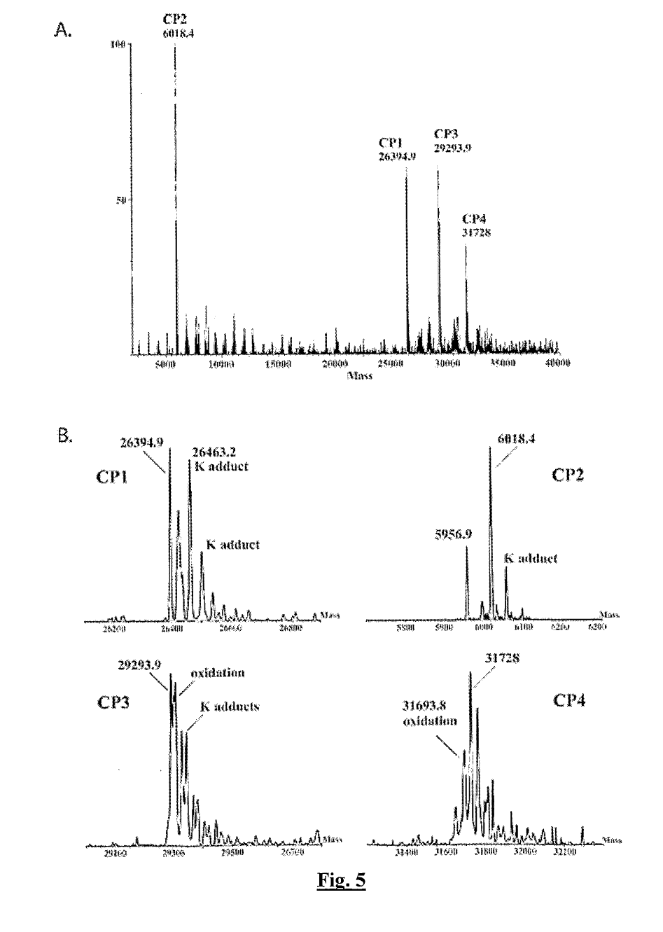

The intact proteins of PnPV were also analyzed by electrospray ionization mass spectrometry (ESI/MS). FIG. 5 shows the neutral molecular-mass spectrum obtained by electrospray ionization of the entire virus capsid (i.e., not digested), in which molecular masses corresponding to intact CP1, CP2, CP3, and CP4 proteins are indicated. The molecular mass of intact CP1 at 26,394.9 Da is consistent with the CP1 sequence beginning with glycine at the N terminus (amino acid 320) and ending with glutamine at the C terminus (amino acid 556). This supports the conclusion from the protein digest analysis that the predicted amino acids 557-573 are not found in the mature CP1 protein. The measured mass of 26,394.9 Da is approximately 33 Da larger than the theoretical mass of 26,361.7 Da. Correspondingly, the observed complexity of higher mass molecular peaks in roughly 32 Da increments suggests that minor-level oxidation or other modifications existed in the protein as analyzed.

The molecular mass of intact CP2, determined to be 6018.4 Da, is in agreement with the theoretical mass of 6019.0 Da (average-isotope composition), with no apparent post-translational modifications present. There is also a protein at 5956.9 Da present that does not result from simple cleavage of any of the CP proteins, and its source was not determined. CP3 yielded an intact molecular mass of 29,293.9 Da, which is consistent with the theoretical mass of 29,294.2 Da, and some degree of oxidation or minor modification of the protein is apparent from the presence of higher mass peaks (e.g., 29,345 Da).

The analysis of intact CP4 shows a molecular mass at 31,693.8 Da (theoretical mass of 31,662.9) that is consistent with an oxidized form of the protein. However, similar to CP1, the complexity of molecular species indicates other modifications of the protein may also be present. Taken together with the unequivocal protein digest data, we conclude that CP4 ends with glycine encoded by codon 1190 at the end of the 2A sequence.

We aimed to identify the carboxy-terminus of a protein containing a 2A site. There were two candidates in the PnPV virion, CP4 and CP1. Identification of peptides by LC/MS/MS with FT-ICR showed that the coding region of CP4 extends to the terminal glycine, encoded by codon 1190, of the 2A sequence. Analysis of full-length virion proteins by ESI/MS indicated that CP4 is encoded by codons 907-1190. The peptide and full-length protein data for CP1 showed that it is encoded by codons 320-556 rather than the expected 320-573. There would have been 17 additional amino acids if all of 2A had been present. PnPV cp1 specifies a potential virally encoded cysteine protease cleavage site ending 17 codons before the 3′ end of a sequence specifying 2A, which likely accounts for the observed truncation.

The selective advantage of a 2A-encoding sequence at the 3′ end of cp1 may permit a step-down in the level of products encoded 3′ as has been observed with reporter constructs using the FMDV 2A sequence [Donnelly et al. 2001a,b]. However, Donnelly et al. did not observe this step-down of expression in the native FMDV polyprotein context, implying that there are important sequence features in addition to 2A that are contained in the 14 amino acids preceding the 2A sequence [Donnelly et al. 2001a]. A step-down is not evident from the proportion of CP1 and CP3 from SDS-PAGE analysis of PnPV virions (data not shown; Wu et al. 2002). It could be that before the viral protease is synthesized, precursor CP1 containing the C-terminal 2A sequence has a nonstructural role, e.g., a role in translation/replication regulation. If so, this form of CP1 would not be detected in this analysis since it would not be expected to be incorporated into virions. A further possibility for 2A function is to cause a pause in translation [Donnelly et al. 2001b] allowing relevant RNA or protein folding(s) to occur.

Analysis of FMDV capsids showed a corresponding viral protease cleavage that removed most of the 2A-encoded sequence at the 3′ end of the terminal coat protein gene with the protein ending with Q [Ryan et al. 1989]. Extrapolating from the now established C terminus and the viral genome sequence encoding 2A, the released peptide is predicted to be (Q)LLNFDLLKLAGDVESNPG(P) (SEQ ID NO. 20), i.e., one amino acid longer than its PnPV counterpart.

These results support the model postulated by Ryan and colleagues [Donnelly et al. 2001b] (now termed StopGo). The model is that a sense codon specifies the C-terminal amino acid of one protein and a 3′ adjacent sense codon specifies a non-methionine N-terminal amino acid of a separate protein. Previous work with a 2A sequence flanked by reporter genes showed that proteolysis is not involved [Ryan and Drew 1994] and that, in a nascent peptide sequence-dependent manner, cotranslational generation of the two products results in relatively more of the upstream-encoded product. The possibility that peptidyl-tRNA containing the upstream encoded product “drops off” from the ribosome prior to hydrolysis has not been directly tested, but how this could happen with retention of tRNA^{Gly} in the A-site for subsequent synthesis of the downstream-encoded product is far from apparent. Based on the intraribosomal peptidyl-tRNA hydrolysis model of Donnelly et al. (2001b), there is only one directly encoded protein residue, proline, which is an imino acid. Its [symbol] dihedral angle is constrained by its pyrrolidine ring to approximately 60°. In the 2A sequence, it is the penultimate amino acid in the nascent chain and would be expected to distort the positioning of the relevant part of the nascent peptide. The C-terminal amino acid is glycine, which forms a flexible peptide bond and is expected to allow the peptide at this position to be less constrained. An understanding of the significance of the properties of these two amino acids and how the key amino acids encoded upstream affect the phenomenon will require detailed structural knowledge. In addition to the importance of these amino acids and the ribosome, the CCA end of tRNA is doubtless also critical [Weinger et al. 2004; Feinberg and Joseph 2006] and can have effects on codon:anticodon dissociation [O'Connor et al. 1993].

Protein synthesis is dependent on reaction of the α-amino group of aminoacyl-tRNA bound to the ribosomal A-site with the ester carbon of peptidyl-tRNA bound in the P-site [Green and Lorsch 2002]. Pro-tRNA^{Pro} acts as a particularly poor nucleophile (as does the glycine counterpart) [Nathans and Neidle 1963; Rychlik et al. 1970]. Continuation of standard protein synthesis in response to an accepted A-site aminoacyl-tRNA involves ribosomal RNA undergoing movements that reorient the ester linkage, making it accessible for attack [Schmeing et al. 2005a,b]. Before the next tRNA is accepted into the A-site, the peptidyl-transfer center positions the ester linkage so that it is not accessible to nucleophilic attack by water with resulting deacylation of peptidyl-tRNA as happens when a release factor is accepted in an A-site containing a stop codon. The exact features that can reprogram the ribosome so there is hydrolysis of peptidyl-tRNA^{Gly}, but with continued synthesis resulting in the proline acylated to the next A-site tRNA and becoming the N-terminal amino acid of a separate chain, are not understood.

Despite the rRNA differences between prokaryotic and eukaryotic ribosomes, other studies in Escherichia coli are pertinent. A strong regulatory pause occurs during translation of E. coli secM mRNA until the N terminus of the nascent chain is “pulled” by the secretory apparatus. The pause is mediated by specific amino acids encoded upstream of the pause site interacting with specific 23S rRNA nucleotides and ribosomal protein L22 that are located at the constriction of the nascent peptide exit tunnel through the large ribosomal subunit [Nakatogawa and Ito 2002]. This interaction signal is transduced to the peptidyl-transfer center [Mitra et al. 2006; Woolhead et al. 2006]. The strong pause occurs with cognate peptidyl-tRNA^{Gly} at the ribosomal P-site and cognate aminoacyl-tRNA^{Pro} accepted in the ribosomal A-site [Muto et al. 2006]. Although the block occurs before transfer of the peptide chain to Pro tRNA^{Pro}, having that tRNA in the A-site is critical for the process [Muto et al. 2006] (and, in addition, its presence inhibits tmRNA accessibility) [Garza-Sanchez et al. 2006]. The specific ribosomal changes in response to the nascent peptide signal in this case do not remove the protection of the ester linkage of the peptidyl-tRNA^{Gly} from P-site nucleophilic attack by water with resulting deacylation. The distinctions that cause deacylation with 2A remain to be determined (uncharged tRNA in the A-site triggers peptidyl-tRNA deacylation but the tRNA in this case is, of course, acylated). The relevant glycine codon in the first 2A-encoding sequence in PnPV RNA is GGA, and in the second it is GGG. In 2A sequences in other genomes (Table 1) or in tested mutants, there is no preferred glycine codon. Since no restriction to GGG codons is evident, the situation is different from drop-off in E. coli, which has been reported to occur preferentially at NGG sequences [Gonzalez de Valdivia and Isaksson 2005].

TABLE 1
The last two codons specifying proline
and glycine of the upstream 2A-encoded
product and the first codon
specifying proline of the downstream
product (PG/P) found in viruses.

| | P | G | P |
|---|---|---|---|
| EMCV-B | CCU | GGA | CCC |
| EMCV-D | CCU | GGA | CCC |
| EMCV-PV21 | CCA | GGG | CCC |
| MENGO | CCC | GGG | CCU |
| Theilovirus TME-GD7 | CCA | GGC | CCU |
| Theilovirus TME-DA | CCA | GGC | CCU |
| Theiler's-like virus | CCA | GGC | CCG |
| Ljungan virus (174F) | CCA | GGC | CCU |
| Ljungan virus (145SL) | CCA | GGU | CCU |
| Ljungan virus (87-012) | CCA | GGC | CCU |
| Ljungan virus (M1146) | CCU | GGG | CCC |
| FMD-C1 | CCU | GGG | CCC |
| FMD-O/SK | CCU | GGG | CCU |
| FMD-SAT2 | CCU | GGG | CCC |

Insect viruses

| CrPV | CCU | GGU | CCU |
|---|---|---|---|
| DCV | CCU | GGA | CCC |
| ABPV | CCU | GGA | CCU |
| IFV | CCU | GGA | CCU |
| TaV | CCC | GGC | CCC |
| APV | CCC | CCU | CCA |
| KBV | CCU | GGA | CCC |
| PnPV | | | |
| (a) | CCC | GGA | CCC |
| (b) | CCU | GGG | CCC |
| Ectropis obliqua | | | |
| picorna-like virus | | | |
| (a) | CCC | GGC | CCC |
| (b) | CCC | GGC | CCA |
| Providence virus | | | |
| (a) | CCU | GGG | CCC |
| (b) | CCC | GGA | CCU |

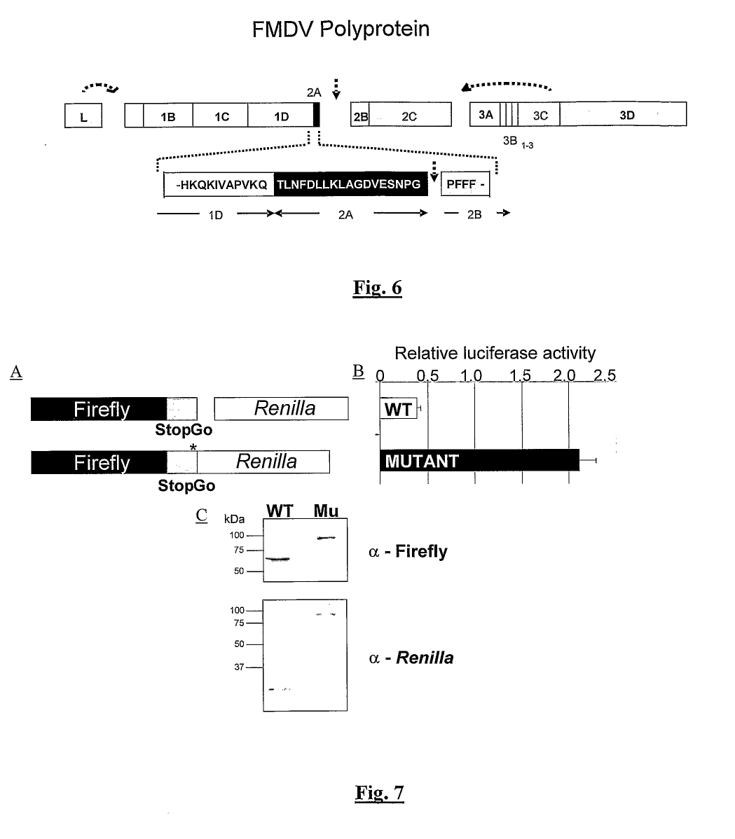

Although FMDV 2A is defined as being only 18 amino acids long, it is part of a [P1-2A] fusion protein when it mediates StopGo translation. We have shown that sequences upstream of 2A (capsid protein 1D) exert an influence on 2A-mediated StopGo translation. Experiments were performed using artificial polyprotein systems. Single ORFs were constructed to encode green fluorescent protein (GFP) linked via the test sequence to β-glucuronidase (GUS) [Donnelly et al, 2001 (a) and (b); Luke et al, 2008]. The processing properties of such recombinant ORFs were analysed using translation systems in vitro. The incorporation of sequences upstream of 2A (along with 2A) showed the functional length of FMDV 2A to be somewhat longer than 18 amino acids. The small amounts of fusion protein observed with the 18 amino acids version and the N-terminal proline residue of 2B (SEQ ID NO. 1-LLNFDLLKLAGDVESNPGP-) was very greatly reduced with a longer (SEQ ID No. 2 33 amino acids: -EARHKQKIVAPVKQTLNFDLLKLAGDV ESNPGP-) version of 2A [Donnelly et al, 2001a]. The data for FMDV 2A concerning the lengths of the sequences required for StopGo translation was confirmed by the analysis of a wide range of 2A-like sequences from other viruses [Donnelly et al 2001a; Luke et al, 2008; Donnelly et al, 1997]. The activity of all the 2A and ‘2A-like’ sequences we have analysed all map to an oligopeptide sequence of 30 amino acids (SEQ ID No. 3-HKQKIVAPVKQTLNFDLLKLAGDVESNPGP as represented schematically in FIG. 6.

Using translation systems in vitro to analyse 2A activity provided a vital clue as to the mechanism. A key feature of 2A-mediated StopGo translation is that sequences upstream of 2A may be synthesised to greater levels than those downstream. As the term implies, translation ‘stops’ (terminates mid ORF) and in a high (but certainly not all) proportion of cases, translation of downstream sequences resumes (‘go’). This imbalance in the synthesis of translation products—derived from a single ORF—together with the determination of the length of the 2A/2A-like sequence required for activity allowed us to formulate a model of the mechanism of StopGo translation.

We propose that the nascent oligopeptide (2A) sequence adopted a helical conformation and interacted with the exit tunnel of the ribosome to influence translation.

We believe that the interaction of 2A with the exit tunnel may also serve to fix the stereochemistry of a tight turn at the base of the helix (-SNPG-) such that the ester linkage between the nascent protein and tRNA^{gly} adopted a conformational space (in the P site of the ribosome) that precluded it form nucleophilic attack by prolyl-tRNA^{pro} in the A site of the ribosome. Site-directed mutagenesis and absolute conservation of this residue (corresponding to the N-terminal proline of FMDV protein 2B) showed that the identity of this residue was an absolute requirement for activity [Donnelly et al 2001a; Luke et al, 2008]. Proline is unique amongst amino acids as the imide nitrogen is part of a ring structure thereby stereochemically constraining the nucleophilic electrons on the imide nitrogen.

The interaction between 2A and the ribosomal exit tunnel also is an absolute requirement for 2A-mediated StopGo translation. It has been shown that the dimensions of the ribosome exit tunnel are such that it can accommodate the nascent protein in a helical conformation, and that this length is ˜40aa [Voss et al, 2006]—consistent with our data mapping the length of the sequences required for 2A activity. We have shown that disruption of this interaction by site-directed mutagenesis either reduces, or abrogates, the activity of 2A [Donnelly et al, 2001a].

The mechanism of action of some small molecule inhibitors of prokaryotic protein synthesis have been elucidated for example, negamycin, binds at a single site formed by highly conserved nucleotides near the cytosolic end of the tunnel [Schroeder et al, 2007]. The binding site of many macrolides, lincosamide, and streptogramin B. antibiotics have been determined ([Tu et al, 2005] and references therein). The macrolide erythromycin, a 14-membered lactone ring containing small molecule, has been reported to initially bind at the lower affinity site at the interface side of the exit tunnel and subsequently at a higher interface site just inside the tunnel [Petropoulos et al, 2008]. It blocks the synthesis of peptides longer than between six to eight amino acids, and expression of the ErmC methyltransferase gene which confers resistance to erythromycin is regulated by ribosome stalling mediated by nascent peptide-erythromycin interactions in the decoding of an ORF in the leader of the methyltransferase mRNA [Mayford and Weisblum, 1990].

Negamycin causes some readthrough of stop codons, that is, at a low frequency the stop codon specifies an amino acid with continued downstream decoding, instead of specifying terminate protein synthesis [Allamand et al, 2008]. For decades it has been known that antibiotics such as the aminoglycoside gentamycin and also paramomycin cause stop codon readthrough, and many of these are now known to act at the ribosomal A-site. Since a subset of patients with many genetic diseases such as cystic fibrosis or Duchenne muscular dystrophy have mutations creating premature stop codons, the ability to cause some readthrough of those stop codons to generate a small amount of full-length protein has been sought. Several studies have focussed on the sequence context of the stop codon for optimal gentamycin action in test sequences ([Howard et al, 2000] and the references therein) or on the variety of compounds that cause readthrough [Thompson et al, 2004]. Several of these compounds were initially studied by pharmaceutical companies because of their inhibition of bacterial protein synthesis, with tolerable effects on mammalian protein synthesis. However, some such as gentamycin and amikacin cause significant levels of stop codon readthrough in certain situations in mammalian systems ([Du et al, 2006] and the references therein). Testing either natural compounds or chemically synthesized derivatives which were identified on the basis of selective anti-bacterial action, for desirable effects on mammalian protein synthesis, is not, however, the only option available. One biotechnology company has developed a compound specifically for the purpose of causing enhanced readthrough of premature termination codons in mammals and it is reported to be more useful that compounds initially selected on the basis of their selective anti-bacterial effects [Du et al, 2008]. However none of these small inhibitor molecules are specifically targeted to the StopGo mechanism.

We envisage that the interaction between a wild-type 2A sequence of a pathogen (such as FMDV) and the exit tunnel of a eukaryotic ribosome could be disrupted by targeted small molecules which may enter the ribosome exit tunnel. We believe that the use of small molecules targeted to this site may disrupt the action of the ribosome and thereby reduce or abrogate 2A-mediated StopGo translation.

The invention will be more clearly understood from the following examples.

### EXAMPLES

### 2A Conserved Amino Acid Sequence

Studies of 2A protein sequences have demonstrated that NPGP (SEQ ID No. 30) is not only a conserved C terminal amino acid sequence but that it is an essential sequence for StopGo translated recoding.

The amino acid sequence DXEXNPGP (SEQ ID No. 28) has been shown to be highly conserved at the C terminal of 2A proteins in which X in position 2 is valine and X in position 4 is serine in FMDV 2A protein. Some mutagenesis studies have been performed which have demonstrated that X in position 2 and 4 cannot be a proline residue as this would disrupt/break the α-helix formed by the 2A protein. Donnelly et al (2001) mutated X in position 4 from serine to isoleucine and from serine to phenylalanine and demonstrated that the FMDV 2A protein retained about 42% and about 39% activity respectively. Hahn and Palmenberg conducted some mutagenesis studies in the EMCV 2A protein in which X in the position 2 is isoleucine and X in the position 4 is threonine. Hahn and Palmenberg demonstrated that mutating the X in position 2 from isoleucine to phenylalanine was not tolerated but that mutating isoleucine to valine was tolerated (it should be noted that in the Mengovirus strain of EMCV, the X in position 2 is valine). Hahn and Palmenberg also demonstrated that mutating X in position 4 from threonine to alanine was well tolerated in EMCV.

Some natural variations of X in position 2 and 4 in 2A proteins have been elucidated as: X in position 2 being valine in TMEV and X in position 4 being methionine in TME V and glutamate in Ljungan virus.

It is anticipated that conservative substitutions of X in position 2 and 4 will also be tolerated. Conservative amino acid substitutions generally maintain (a) the structure of the polypeptide backbone in the area of the substitution, for example, as a beta sheet or alpha helical conformation. (b) the charge or hydrophobicity of the molecule at the site of the submission, and/or (c) the bulk of the side chain. Table 2 below lists the conventionally accepted conservative substitutions.

TABLE 2
Conservative substitutions of amino acids

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Donnelly et al (2001) investigated the effects of substituting the residues D, E and N in the FMDV 2A sequence D-V-E-S-N-P-G-P (SEQ ID No. 31) and found that mutating D to E or Q abolished 2A activity; mutating E to N or G resulted in no activity and 56% activity of 2A respectively; and mutating N to H, E, or Q resulted in a reduction in 2A activity to 31%, 19% and 10% respectively.

Likewise, Hahn and Palmenberg studied the effects of substituting the residues D, E and N in the EMCV 2A sequence D I E T N P G P (SEQ ID No. 32) and found that mutating D to H or N resulted in no activity or limited (less than about 10%) activity respectively; mutating E to D reduced the level of EMCV 2A activity; and mutating N to K resulted in a reduction (to less than about 10%) in EMCV 2A activity.

Whilst the D V E S N P G P (SEQ ID No. 31) sequence is the preference for 2A StopGo activity, and notwithstanding the above mentioned mutagenesis studies, it is envisaged that conservative substitution of one or more of the residues D, V, E, S or N will be tolerated in the screening methods/assays described herein.

### Generation of Sequences Encoding PGP (SEQ ID No. 29)

Sequences encoding the amino acid sequence PGP (SEQ ID No. 29) may be generated for use in the assays and or screening methods described herein.

Generation of a sequence encoding on amino acid sequence comprising PGP (SEQ ID No. 29) may be performed using cloning or synthesis techniques. For example, sequences encoding the amino acid sequence comprising PGP (SEQ ID No. 29) can be cloned by annealing two complimentary oligonucleotides with flanking restriction enzyme sites which can then be digested and ligated into a suitably digested plasmid. Alternatively, double stranded DNA encoding the amino acid residues could be commercially synthesised to incorporate flanking restriction sites and cloned into a suitably designed plasmid. The generation of sequences encoding amino acids PGP (SEQ ID No. 29) can be performed using conventional cloning or synthesis techniques known to a person skilled in the art.

It will be appreciated that an amino acid sequence comprising PGP (SEQ ID No. 29) may contain additional amino acid residues. Preferably, the additional amino acid residues will be located upstream of the PGP (SEQ ID No. 29) sequence. We have found that the screening/assay methods described herein work best when there are at least ten amino acids upstream of the PGP sequence (SEQ ID No. 29).

### Inhibitor Screens

Inhibition of the StopGo translational effect by a small molecular inhibitor will disrupt ribosome functioning: for example, the inhibitor may interfere with the interaction between 2A and the ribosome exit tunnel which results in a pause in the processivity of the ribosome [Weinger and Strobel; 2006]. Disruption of this interaction may result in normal peptide bond formation, producing a higher proportion of the full-length translation product. Alternatively, the inhibitor may interrupt the transmission of conformational change signals mediated by StopGo action to the site of peptidyl transfer or affect the peptidyl-transferase site or even affect the positioning of the ribosomal A-site tRNA^{Pro}.

Whilst the inhibitor/compound screens described in the non-limiting Examples below utilise plasmids, it will be appreciated that PCR techniques may be used to generate a linear fragment of DNA that encodes an amino acid sequence comprising SEQ ID No. 29, and that this DNA sequence could be used in place of the plasmid for an in vitro translation assay. If PCR techniques are used instead of plasmid techniques the PCR fragment (DNA) will have to be amplified with primers incorporating a suitable promoter prior to translation. Amplifying the PCR fragment can be achieved by inserting a promoter at the 5′ end of the fragment. A suitable promoter such as T7 or SP6 can be inserted into the fragment by designing the forward primer with a promoter sequence at the 5′ end.

### Example 1

### Cell-Free Screen In Vitro

Activity of putative inhibitors will be assayed by the use of cell-free translation systems. Rabbit reticulocyte lysates (RRLs) will be programmed with a RNA encoding a single, long open reading frame comprising green fluorescent protein (GFP), 2A and β-glucuronidase (GUS) [Donnelly et al, 2001b]. 2A-mediated StopGo translation produces three translation products;
(i) 97 kDa (full-length translation product),
(ii) a fusion protein comprising [GFP-2A]—product by translation terminating at the C-terminus of 2A and
(iii) GUS—produced by translation recommencing at the N-terminus of GUS.

This plasmid construct (pGFP2AGUS) encodes a version of 2A comprising the C-terminal glutamine residue (Q) of capsid protein 1D, FMDV 2A and the N-terminal proline residue (P) of FMDV protein 2B (-QLLNFDLLKLAGDVESNPGP- [SEQ ID No. 20]). Using this version of 2A, the full-length translation product [GFP_{—}2A-GUS] comprises ˜10% of the total translation products. Translation profiles are obtained by programming translation extracts with the plasmid construct or RNA transcribed from such plasmids and the synthetic reaction monitored by the incorporation of the radio-labelled amino acid ³⁵S-methionine into translation products. The in vitro translation reaction is then analysed using SDS polyacrylamide gel electrophoresis (10% SDS-PAGE) and the distribution of radioactivity determined by phosphorimaging [Donnelly et al, 2001b]. In this manner, the relative abundance of each of the three translation products ([GFP-2A-GUS]—100 kDa, GUS-70 kDa and [GFP-2A]—30 kDa) can be determined. The methionine content of all products is known (GUS-12:2A=0, GFP=6), the relative abundance of each translation product can be calculated and, therefore, putative inhibitors of this specific step in the mechanism of StopGo translation identified. Compounds/molecules suitable for inhibiting StopGo translation can be identified by the presence of a full length translation product.

Translation reactions (7.5 μl) will be supplemented with inhibitors with a final concentration spanning the nano-to micomolar range. After incubation at 37° C. for 40 min, reactions will be analysed by SDS-PAGE and the distribution of radiolabel determined by phosphorimaging.

### Example 2

### Cell-Free Screen In Vitro

The coupled transcription/translation rabbit reticulocyte lysates (RRLs) programmed with pGFP2AGUS and the synthetic reaction monitored by the incorporation of a nonradioactive label such as FluoroTect™ Green_{Lys}. This in vitro translation labeling system allows the fluorescent labeling of in vitro translation products through the use of a modified charged lysine transfer RNA labeled with the fluorophore BODIPY®-FL. Using this system, fluorescently labeled lysine residues are incorporated into nascent proteins during translation. The fluorescent lysine is added to the translation reaction as a charged epsilon-labeled fluorescent lysine-tRNA complex (FluoroTect™ Green_{Lys} tRNA) rather than a free amino acid. Translation reactions (7.5 μl) will be supplemented with inhibitors with a final concentration spanning the nano-to micomolar range. After incubation at 37° C. for 40 min. the in vitro translation reaction is then analysed using SDS polyacrylamide gel electrophoresis (10% SDS-PAGE) and the distribution of labelled proteins determined directly “in-gel” using a laser-based fluorescent scanner. In this manner, the relative abundance of each of the three translation products ([GFP-2A-GUS]—100 kDa, GUS-70 kDa and [GFP-2A]—30 kDa) can be determined. Compounds/molecules suitable for inhibiting StopGo translation can be identified by the presence of a full length translation product.

### Example 3

### Dual Reporter Cell-Based Screen

A dual reporter system that uses fluorescent encoding proteins may be useful as a cell based screen and unlike enzyme encoding reporter systems (such as the lucifersase system described in Example 4) as there is no requirement for enzymatic activity and hence no expensive enzyme substrates. In addition, fluorescent proteins can be detected in living cells. One example of a fluorescent dual reporter is the fusion of cyan fluorescent protein (CFP: acceptor) to the N-terminus of WT or mutant StopGo with yellow fluorescent protein (YFP: donor) fused to the C-terminus. When the two fluorescent proteins are dissociated (such as during StopGo) the donor (YFP) emission is detected using a standard fluorimeter upon the donor excitation. On the other hand, when the donor and acceptor are in close proximity (such as when StopGo is inhibited by a small molecule), the acceptor emission is predominantly observed because of the intermolecular fluorescence resonance energy transfer (FRET) from the donor to the acceptor. The fluorescent protein pairs may be any combination available that have well separated excitation and emission maxima (e.g. green fluorescent protein together with either cherry red or tomato red fluorescent proteins).

A construct encoding artificial polyproteins comprising yellow fluorescent protein (YFP), 2A and cyan fluorescent protein (CFP) has been created (plasmid pPDF20) and expression analysed by transfection of cells [deFelipe and Ryan, 2004]. This results in the co-expression of both fluorescent proteins in the cytoplasm (with passive transport into the nucleus) of transfected cells. When the construct is modified by the insertion of a co-translational signal sequence (GalT; plasmid pPDF19) immediately downstream of 2A, the first protein in the polyprotein (YFP) is located in the cytoplasm, whilst the second protein (CFP-downstream of 2A, produced as a discrete translation product) is now targeted to the Golgi apparatus [deFelipe and Ryan, 2004]. This produces a clear separation of these proteins within infected cells.

Fluorescence resonance energy transfer (FRET) occurs when excitation of the donor (cyan) molecule leads to emission from the acceptor (yellow) molecule. This only occurs if the two proteins are close enough for energy transfer to occur [Pollock and Helm, 1999; http://www.lamondlab.com/pdf/FRET.pdf]. When YFP and CFP are proximal, therefore, a FRET signal can be detected, but not when they are spatially separated. Cellular expression of pPDF19 will not result in FRET, since the two proteins are spatially separated. Inhibition of the 2A-mediated StopGo translation reaction would, however, result in a [YFP-2A-CFP] fusion protein (co-localised in the cytoplasm) such that a FRET signal would now appear. This provides a cell-based screening system suitable for high-throughput screening of putative inhibitors of 2A-mediated StopGo translation.

### Example 4

### Dual-Luciferase Assay

Two constructs for monitoring the effect of putative inhibitory compounds for in vitro and in vivo experiments have been constructed and tested. These reporter constructs fuse, in one instance the firefly luciferase encoding sequence to the 5′ end of a 2A-encoding sequence (SEQ ID No. 3-HKQKIVAPVKQTLNFDLLKLAGDVESNPGP) which is fused to the 5′ end of a Renilla luciferase encoding sequence. and in another instance fuses the Renilla luciferase encoding sequence to the 5′ end of a 2A-encoding sequence (SEQ ID No. 3-HKQKIVAPVKQTLNFDLLKLAGDVESNPGP) which is fused to the 5′ end of a firefly luciferase encoding sequence such that translation of this reporter after transfection into human embryonic kidney cells (HEK-293) produced three proteins: a firefly-Renilla fusion protein with both luciferase activities, a firefly only protein with firefly luciferase activity and a Renilla protein with Renilla luciferase activity. The differential activity of these proteins can be monitored simultaneously using the dual luciferase assay.

In the dual-luciferase assay the activities of firefly and Renilla luciferases are measured sequentially from a single sample. The firefly luciferase reporter is measured first by adding its substrate (Luciferin) to generate a “glow-type” luminescent signal. After quantifying the firefly luminescence the Renilla luciferase reaction is initiated by simultaneously adding a quencher and the Renilla substrate (coelenterazine) to the same tube. Both reporters yield linear assays with subattomole sensitivities and no endogenous activity of either reporter in experimental host cells.

Additionally, firefly and Renilla proteins can be visualized by Western Blotting with Firefly or Renilla antibodies to allow further quantitation of StopGo activity.

Sequences encoding wild-type (WT) and mutant (non-functional) FMDV StopGo cloned into a dual luciferase reporter plasmid (Grntzmann et al. 1998) such that Renilla luciferase is N-terminal and Firefly luciferase is C-terminal of WT and mutant StopGo. Transfection of these constructs into HEK-293 cells and comparison of the Firefly/Renilla ratio between WT and mutant constructs indicated a 2-fold difference. Although this 2-fold difference is sufficient to discern inhibition of StopGo (which would have a Firefly/Renilla ratio similar to the non-functional mutant), a larger difference is desirable to more readily discern complete inhibition from partial inhibition. A reverse reporter construct was made in which Firefly luciferase was at the N-terminal and Renilla luciferase was at the C-terminal of WT and mutant StopGo. Transfection of these constructs into HEK-293 cells and comparison of the Firefly/Renilla ratio between WT and mutant constructs resulted in a 4-5 fold difference (FIG. 7).

Luciferase levels can be analysed using Western Blotting or their activities assayed using one of the following methods:

### A)

add a substrate for the first luciferase enzyme;
quantify the luminescent signal produced by the first luciferase enzyme;
add a quencher to quench the first luciferase enzyme;
add a substrate for the second luciferase enzyme; and
quantify the luminescent signal produced by the second luciferase enzyme.

The first luciferase enzyme may be firefly luciferase and the second luciferase enzyme may be renilla luciferaces. Coelenterazine is a suitable substrate for firefly luciferase and luciferin is a suitable substrate for the Renilla luciferase. The quencher and the substrate for the second luciferase enzyme may be added simultaneously.

### B)

split the translation system into two sub populations;
add a substrate for the first luciferase enzyme to the first sub populations;
quantify the luminescent signal produced by the first luciferase enzyme;
add a substrate for the second luciferase enzyme to the second sub population; and
quantify the luminescent signal produced by the second luciferase enzyme.

The translation system may be a cell based translation system and the cells may be broken before splitting the system into two sub populations. The first luciferase enzyme may be firefly luciferase and the second luciferase enzyme may be Renilla luciferase. Coelenterazine is a suitable substrate for firefly luciferase and luciferin is a suitable substrate for the Renilla luciferase. The substrates for the first and second luciferase enzymes may be added simultaneously.

### Example 5

### Putative Bovine and Human StopGo Sequences

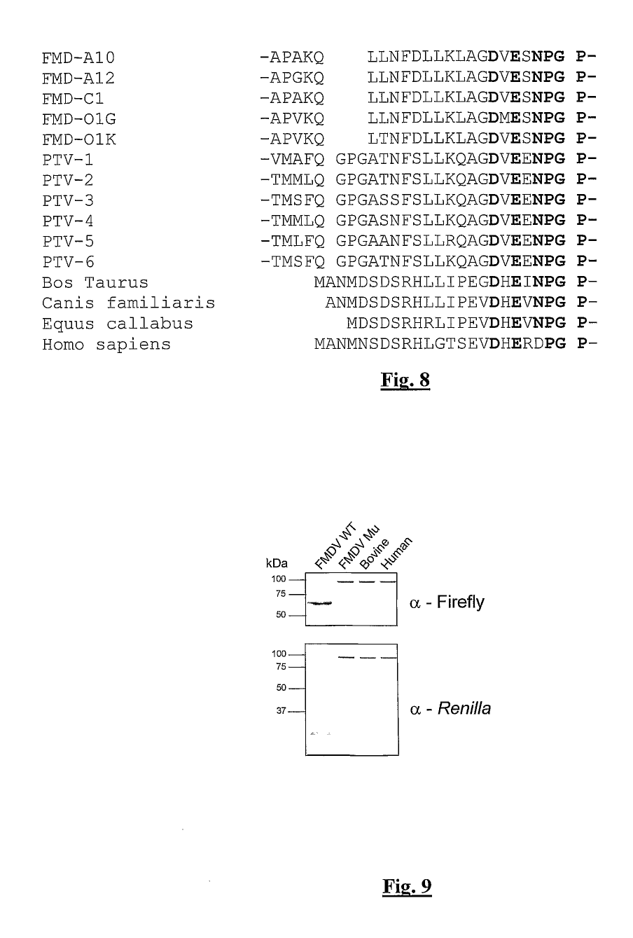

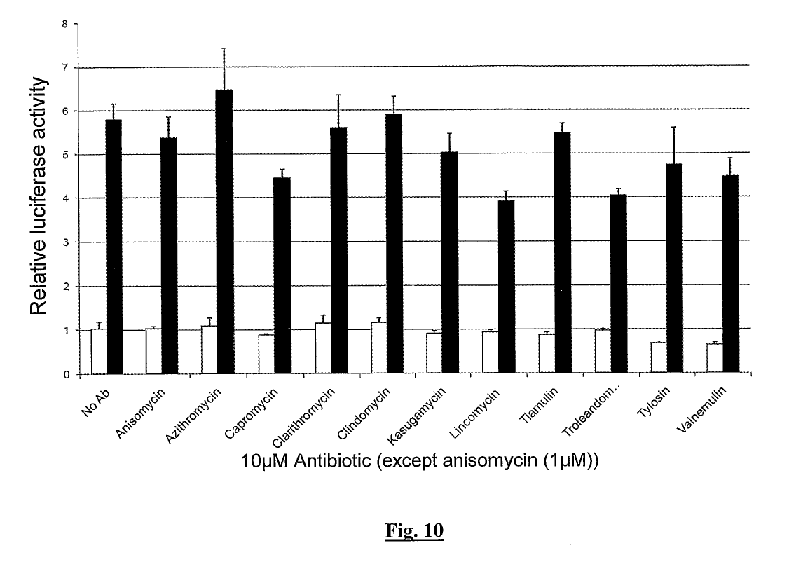

Homology searches of the available protein databases with the StopGo signature motif revealed a previously unnoticed putative bovine StopGo sequence within a gene encoding an amino acid transporter (FIG. 8). The success of our anti-viral approach to specifically target FMDV relies on there being no host targets. Therefore it was necessary to test whether this putative StopGo sequence encoded within the bovine genome was active. To address this we cloned the bovine StopGo encoding sequence and the human homolog of this gene into the dual luciferase vector (Grentzmann et al, 1998) as follows: Sequences encoding the putative bovine (ANMDSDSRHLLIPEGDHEINPG-P) (SEQ ID No. 33) or human (ANMNSDRSRHLGTSEVDHERDPG-P) (SEQ ID No. 34) StopGo were cloned into p2-Luc and then the plasmid DNA from 2 individual clones was isolated and used to transfect in quadruplicate HEK-293 cells as previously described. 24 hours after transfection all cells were lysed and proteins were analysed by Western Blotting in duplicate (FIG. 9) using either anti-Renilla or anti-firefly separately. Detection of a single protein of ˜100 kDa that is recognised by both antibodies rather than two separate proteins indicates that the bovine and human putative StopGo sequences are inactive. These constructs were transfected into HEK-293 cells and assayed by both the dual luciferase assay and by western blotting with Firefly and Renilla luciferase antibodies (FIG. 9.). When compared to both WT active FMDV StopGo and Mu inactive FMDV StopGo, it is clear that both bovine and human StopGo sequences give results that are almost identical to the inactive FMDV indicating that the bovine and human sequences are inactive.

### Example 6

### Assaying the Effect of Known Ribosomal Targeting Antibiotics on StopGo

Using the reporter system described in Example 4 above that gave 4-5 fold ratio (Firefly luciferase at the N terminal and Renilla luciferase at the C terminal) we tested several known antibiotics for any possible effect on StopGo action. HEK-293 cells were transfected with either WT or Mutant FMDV sequences for 4 hours before adding a serial dilution of the test antibiotics ranging from 0.1-1000 μM for 48 hours. After 48 hours cells were assayed by the dual luciferase assay. The following antibiotics were tested—anisomycin, azithromycin, capromycin, clarithromycin, clindomygin, gentamycin, kasugamycin, lincomycin, tiamulin, troleandomycin, tylosin, valnemulin (FIG. 10). The ratio of Renilla/firefly was compared between the cells transfected with the WT StopGo and the mutant StopGo. Antibiotics that have any effect on StopGo would be expected to alter the Renilla/firefly ratio of the WT StopGo expressing construct. None of the antibiotics tested had any effect on the Renilla/firefly ratio of the WT StopGo expressing construct at sub-toxic concentrations, indicating that they do not affect StopGo under these conditions. This strongly suggests that there must be a certain level of specificity required to affect StopGo.

The invention is not limited to the embodiment hereinbefore described, with reference to the accompanying drawings, which may be varied in construction and detail.

### REFERENCES

Allamand, V., Bidou, L., Arakawa, M., Floquet, C., Shiozuka, M., Paturneau Jonas, M., Gartioux, C., Butler-Browne, G. S., Mouly, V., Rousset, J. P., Matsuda, R., Ikeda, D. and Guicheney, P. 2008. Drug-induced readthrough of premature stop codons leads to the stabilization of laminin alpha2 chain mRNA in CMD myotubes. J. Gene Med. 10, 217-224.
Atkins, J. F., Wills, N. M., Loughran, G., Wu, C-Y., Parsawar, K. Ryan, M. D., Wang, C. H. & Nelson, C. C. 2007. A case for “StopGo”: Reprogramming translation to augment codon meaning of GGN by promoting unconventional termination (Stop) after addition of glycine and then allowing continued translation (Go). RNA 13, 1-8.
Batson S, Rundell K. Proteolysis at the 2A/2B junction in Theiler's murine encephalomyelitis virus. Virology. 1991 April; 181(2):764-7.
Blobel, G, and Sabatini, D. D. 1970. Controlled proteolysis of nascent polypeptides in rat liver cell fractions, J. Cell Biol. 45, 130-145.
Cao, J. and Geballe, A. P. 1996. Coding sequence-dependent ribosomal arrest at termination of translation. Mol. Cell. Biol. 16: 603-608.
Cruz-Vera L R, Gong M, Yanofsky C (2006) Changes produced by bound tryptophan in the ribosome peptidyl transferase center in response to TnaC, a nascent leader peptide. Proc Natl Acad Sci USA 103, 3598-3603.
de Felipe, P. Hughes, L. E. Ryan, M. D. & Brown, J. D. 2003. Co-translational, intraribosomal cleavage of polypeptides by the foot-and-mouth disease virus 2A peptide. J. Biol. Chem. 278, 11441-11448.
de Felipe, P & Ryan, M. D. (2004) Targeting of proteins derived from self-processing polyproteins containing multiple signal sequences. Traffic 5, 616-626.
Donnelly, M., Gani, D., Flint, M., Monoghan, S. & Ryan, M. D. 1997. The cleavage activity of aphtho- and cardiovirus 2A proteins. J. Gen. Virol. 78, 13-21.
Donnelly, M. L. L., Hughes, L. E., Luke, G., Li, X., Mendoza, H., ten Dam, E., Gani, D. & Ryan, M. D. 2001a. The ‘cleavage’ activities of FMDV 2A site directed mutants and naturally-occurring ‘2A-Like’ sequences. J. Gen. Virol. 82, 1027-1041.
Donnelly, M. L. L., Luke, G., Mehrotra, A., Li, X., Hughes. L. E., Gani. D. & Ryan, M. D. 2001b. Analysis of the aphthovirus 2A/2B polyprotein ‘cleavage’ mechanism indicates not a proteolytic reaction, but a novel translational effect: a putative ribosomal ‘skip’. J. Gen. Virol. 82, 1013-1025.
Du, M., Keeling, K. M., Fan, L., Liu, X., Kovacs, T., Sorscher, E. And Bedwell, D. M. 2006. Clinical doses of amikacin provide more effective suppression of the human CFTR-G542X stop mutation than gentamicin in a transgenic CF mouse model. J. Mol. Med. 84, 573-582.
Du, M., Liu, X., Welch, E. M., Hirwat, S., Peltz, S. W. and Bedwell, D. M. 2008. PTC124 is an orally bioavailable compound that promotes suppression of the human CFTR-G542X nonsense allele in a CF mouse model. Proc. Natl. Acad. Sci. USA 105, 2064-2069.
Feinberg, J. S. and Joseph, S. 2006. A conserved base pair between tRNA and 23S rRNA in the peptidyl transferase center is important for peptide release. J. Mol. Biol. 364: 1010-1020.
Garza-Sánchez, F., Janssen, B. D., and Hayes, C. S. 2006. Prolyl-tRNA^{Pro} in the A site of secM-arrested ribosomes inhibits the recruitment of transfer-messenger RNA. J. Biol. Chem. 281: 34258-34268.
Gonzalez de Valdivia, E. I. and Isaksson, L. A. 2005. Abortive translation caused by peptidyl-tRNA drop-off at NGG codons in the early coding region of mRNA. FEBS J. 272: 5306-5316.
Green, R. and Lorsch, J. R. 2002. The path to perdition is paved with protons. Cell 110, 665-668.
Grentzmann G, Ingram J A, Kelly P J, Gesteland R F, Atkins J F, 1998. A dual-luciferase reporter system for studying recoding signals. RNA 4: 479-486
Hahn, H. and Palmenberg, A. C. 1996. Mutational analysis of the encephalomyocarditis virus primary cleavage. J. Virol. 70: 6870-6875.
Hellen, C. U., Krausslich, H. G., and Wimmer, E. 1989. Proteolytic processing of polyproteins in the replication of RNA viruses. Biochemistry 28: 9881-9890.
Heras, S. R., Thomas, M. C., Garcia-Canadas. M., de Felipe, P., Garcia-Pérez, J. L., Ryan, M. D., and Lopez, M. C. 2006. L1Tc non-LTR retrotransposons from Trypanosoma cruzi contain a functional viral-like self-cleaving 2A sequence in frame with the active proteins they encode. Cell. Mol. Life Sci. 63: 1449-1460.
Howard, M. T., Shirts, B. H., Petros, L. M., Flanigan, K. M., Gesteland, R. F. and Atkins, J. F. 2000. Sequence specificity of aminoglycoside-induced stop condon readthrough: potential implications for treatment of Duchenne muscular dystrophy. Ann Neurol. 48, 164-169.
Isawa, H., Asano, S., Sahara, K., Iizuka, T., and Bando, H. 1998. Analysis of genetic information of an insect picorna-like virus, infectious flacherie virus of silkworm: Evidence for evolutionary relationships among insect, mammalian and plant picoma (-like) viruses. Arch. Virol. 143: 127-143.
Jackson R J. A detailed kinetic analysis of the in vitro synthesis and processing of encephalomyocarditis virus products. Virology. 1986 February; 149(1):114-27.
Lu, J., Zhang, J., Wang, X., Jiang, H., Liu, C., and Hu, Y. 2006. In vitro and in vivo identification of structural and sequence elements in the 5′ untranslated region of Ectropis ohliqua picorna-like virus required for internal initiation. J. Gen. Virol. 87: 3667-3677.
Luke, G. A., de Felipe, P., Lukashev, A., Kallioinen, S. E., Bruno, E. A. & Ryan, M. D. 2008. The occurrence, function, and evolutionary origins of ‘2A-like’ sequences in virus genomes. J. Gen. Virol. (accepted for publication).
Malkin, L. I. and Rich, A. 1967. Partial resistance of nascent polypeptide chains to proteolytic digestion due to ribosomal shielding. J. Mol. Biol. 26, 329-346.
Mayford, M. and Weisblum, B. 1990. The ermC leader peptide: amino acid alterations leading to differential efficiency of induction by macrolide-lincosamide-streptogramin B antibiotics. J. Bacteriol. 172, 3772-3779.
Milligan, R. A. and Unwin, P. N. 1986. Location of exit channel for nascent protein in 80S ribosome, Nature 319.693-695.
Mitra, K., Schaffitzel, C., Fabiola, F., Chapman, M. S., Ban, N. And Frank, J. 2006. Elongation arrest by SecM via a cascade of ribosomal RNA rearrangements. Mol. Cell 22, 533-543.
Moscufo, N., Simons, J., and Chow, M. 1991. Myristoylation is important at multiple stages in poliovirus assembly. J. Virol. 65: 2372-2380.
Muto, H., Nakatogawa, H., and Ito, K. 2006. Genetically encoded but nonpolypeptide prolyl-tRNA functions in the A site for secM-mediated ribosomal stall. Mol. Cell 22: 545-552.
Nakatogawa, H. and Ito, K. 2002. The ribosomal exit tunnel functions as a discriminating gate. Cell 108, 629-636.
Nathans, D. and Neidle, A. 1963. Structural requirements for puromycin inhibition of protein synthesis. Nature 197: 1076-1077.
Nicklin, M. J. H., Toyoda, H., Murray, M. G., and Wimmer, E. 1986. Proteolytic processing in the replication of polio and related viruses. Biotechnology (N.Y.) 4: 33-42.
O'Connor, M., Wills, N. M., Bossi, L., and Gesteland, R. F. 1993. Functional tRNAs with altered 3′ ends. EMBO J. 12: 2559-2566.
Palmenberg A C, Rueckert R R. Evidence for intramolecular self-cleavage of picornaviral replicase precursors. J Virol. 1982 January; 41(1):244-9.
Palmenbery A C, Parks G D, Hall D J, Ingraham R H, Seng T W, Pallai P V. Proteolytic processing of the cardioviral P2 region: primary 2A/2B cleavage in clone-derived precursors. Virology. 1992 October; 190(2):754-62.
Petropoulos, A. D., Kouvela, E. C., Dinos, G. P. and Kalpaxis, D. L. 2008. Stepwise Binding of Tylosin and Erythromycin to Escherichia coli Ribosomes, Characterized by Kinetic and Footprinting Analysis. J. Biol. Chem. 283, 4756-4765.
Pollock, B. A., and R. Heim. 1999. Using GFP in FRET-based applications. Trends Cell Biol. 9:57-60.
Raney, A., Law, G. I., Mize, G. J. and Morris, D. R. 2002. Regulated translation termination at the upstream open reading frame in S-adenosylmethionine decarboxylase mRNA, J. Biol. Chem. 277, 5988-5994.
Reuer, Q., Kuhn, R. J., and Wimmer, E. 1990. Characterization of poliovirus clones containing lethal and nonlethal mutations in the genome-linked protein VPg. J. Virol. 64: 2967-2975.
Richards, O. C., Spagnolo, J. F., Lyle, J. M., Vleck, S. E., Kuchta, R. D., and Kirkegaard, K. 2006. Intramolecular and intermolecular uridylyation by poliovirus RNA-dependent RNA polymerase. J. Virol. 80: 7405-7415.
Robertson, B. H., Grubman, M. J., Weddell, G. N., Moore, D. M., Welsh, J. D., Fischer, T., Dowbenko, D. J., Yansura, D. G., Small, B. & Kleid, D. G. 1985. Nucleotide and amino acid sequence coding for polypeptides of foot-and-mouth disease virus type A12. J. Virol. 54, 651-660.
Rueckert, R. R. 1990. Virology, 2nd ed. (eds. B. M. Fields et al.). Raven Press, New York.
Ryan, M. D., Belsham, G. J. & King, A. M. Q. 1989. Specificity of substrate-enzyme interactions in foot-and-mouth disease virus polyprotein processing. Virology 173, 35-45.
Ryan, M. D., King, A. M. Q. & Thomas, G. P. 1991a, Cleavage of foot-and-mouth disease virus polyprotein is mediated by residues located within a 19 amino acid sequence. J. Gen. Virol. 72, 2727-2732.
Ryan, M. D. & Drew, J. 1994. Foot-and-mouth disease virus 2A oligopeptide mediated cleavage of an artificial polyprotein. EMBO J. 11(4), 928-933.
Ryan, M. D. & Flint, M. 1997. Virus-encoded proteinases of the picornavirus super-group. J. Gen. Virol. 78, 699-723.
Ryan, M. D., Donnelly, M. L. L., Lewis, A., Mehrotra, A. P., Wilkie, J. & Gani, D. 1999b. A model for non-stoichiometric, co-translational protein scission in eukaryotic ribosomes. Bioorganic Chem. 27, 55-79.
Ryan, M. D., Luke, G., Hughes, L. E., Cowton, V. M., Dam, E. T., Gani, D., Xuejun, L., Donnelly, M. L. L., Mehrotra, A., and Gani, D. (2002). The Aphto- and Cardiovirus “Primary” 2A/2B polyprotein “cleavage.” In Molecular biology of picornaviruses (eds. B. L. Semler and E. Simmer), pp. 213-223. ASM Press, Washington, D.C.
Rychlík, I., \check{C}erná, J., Chládek, S., Pulkrábek, P., and \check{Z}emli\check{c}ka. J. 1970. Substrate specificity of ribosomal peptidyl transferase. Eur. J. Biochem. 16: 136-142.
Sasaki, J. and Nakashima, N. 2000. Methionine-independent initiation of translation in the capsid protein of an insect RNA virus. Proc. Natl. Acad. Sci. 97: 1512-1515.
Schmeing, T. M., Huang, K. S., Strobel, S. A. and Steitz, T. A. 2005a. An induced-fit mechanism to promote peptide bond formation and exclude hydrolysis of peptidyl-tRNA. Nature 438, 520-524.
Schmeing, T. M., Huang, K. S., Kitchen, D. E., Strobel, S. A. and Steitz, T. A. 2005b. Structural insights into the roles of water and the 2′ hydroxyl of the P-site tRNA in the peptidyl transferase reaction. Mol. Cell. 20, 437-448.
Schroeder, S. J., Blaha, G. And Moore, P. B. 2007. Negamycin binds to the wall of the nascent chan exit tunnel of the 50S ribosomal subunit. Antimicrob. Agents Chemother. 51, 4462-4465.
Thompson, J., Pratt, C. A. and Dahlberg, A. E. 2004. Effects of a number of classes of SOS inhibitors on stopcodon readthrough during protein synthesis. Antimicrob. Agents Chemother. 48, 4889-4891.
Tu, D., Blaha, G., Moore, P. B. and Steitz, T. A. 2005. Structures of MLS_{B}K antibiotics bound to mutated large ribosomal subunits provide a structural explanation for resistance. Cell 121, 257-270.
Voss, N. R., Gerstein, M., Steitz, T. A. & Moore, P. B. 2006. The geometry of the ribosomal polypeptide exit tunnel. J. Mol. Biol. 360, 893-906.
Wang, X., Zhang, J., Lu, J., Yi, F., Liu, C., and Hu, Y. 2004. Sequence analysis and genomic organization of a new insect picorna-like virus, Ectropis obliqua picorna-like virus, isolated from Ectropis obliqua. J. Gen. Virol. 85: 1145-1151.
Wang, Z., Fang, P. and Sachs, M. S. 1998. The evolutionarily conserved eukaryotic arginine attenuator peptide regulates the movement of ribosomes that have translated it, Mol. Cell. Biol. 15, 7528-7536
Weinger, J. S., Parnell, K. M., Dorner, S., Green, R., and Strobel, S. A. 2004. Substrate-assisted catalysis of peptide bond formation by the ribosome. Nat. Struct. Mol. Biol. 11: 1101-1106.
Weinger, J. S, and Strobel, S. A. 2006. Participation of the tRNA A76 hydroxyl groups throughout translation. Biochem. 45, 5939-5948.
Woolhead, C. A., Johnson, A. E. and Bernstein, H. D. 2006. Translation arrest requires two-way communication between a nascent polypeptide and the ribosome. Mol. Cell 22, 587-598.
Wu, C. Y., Lo, C. F., Huang, C. J., Yu, H. T., and Wang, C. H. 2002. The complete genome sequence of Perina nuda picorna-like virus, an insect-infecting RNA virus with a genome organization similar to that of the mammalian picornaviruses. Virology 294: 312-323.
Yonath, A., Leonard, K. R. and Wittmann. H. G. 1987. A tunnel in the large ribosomal subunit revealed by three-dimensional image reconstruction, Science 236, 813-816.
http://www.lamondlab.com/pdf/FRET.pdf

1-69. (canceled)

70. A method of screening compounds or molecules comprising the steps of:
translating a sequence encoding the amino acid sequence comprising SEQ ID No. 29 in a translation system in the presence of a test compound or molecule; and
analysing the translation product(s) for the presence of one or more of
a) a peptide comprising the amino acids Pro-Gly at the C terminus and a peptide comprising the amino acid Pro at the N terminus; or
b) a peptide comprising the amino acid sequence of
SEQ ID No. 29

71. The method as claimed in claim 70 wherein the sequence encodes the amino acid sequence comprising SEQ ID No. 30.

72. The method as claimed in claim 71 wherein the amino acid in position 1 is selected from the group comprising asparagine, histidine, glutamate, glutamine and lysine.

73. The method as claimed in claim 70 wherein the amino acid in position 1 is asparagine.

74. The method as claimed in claim 70 wherein the sequence encodes the amino acid sequence comprising SEQ ID No. 35, the amino acid in position 1 may be selected from the group comprising aspartate and asparagine, the amino acid in position may be selected from the group comprising valine and isoleucine, the amino acid in position 3 may be selected from the group comprising glutamate, aspartate and glycine, the amino acid in position 4 may be selected from the group comprising serine, isoleucine, phenylalanine, threonine, alanine, glutamate, and methionine, the amino acid in position 5 may be selected from the group comprising asparagine, histidine, glutamate, glutamine and lysine.

75. The method as claimed in claim 74 wherein the sequence encodes the amino acid sequence comprising SEQ ID No. 31.

76. The method as claimed in claim 70 wherein the sequence encodes the amino acid sequence comprising SEQ ID No. 1, or SEQ ID No. 2.

77. The method as claimed in claim 70 comprising the step of cloning a sequence encoding the amino acid sequence comprising SEQ ID No. 29 into a plasmid prior to the step of translating the sequence.

78. The method as claimed in claim 70 wherein the sequence is translated in a cell free system, the translation system may be a rabbit reticulate lysate system, the translation system may be a coupled transcription/translation system.

79. The method as claimed in claim 70 wherein the sequence is translated in a cell based system.

80. The method as claimed in claim 70 wherein the sequence comprises a detection tag.

81. The method as claimed in claim 80 wherein the detection tag is located downstream of the sequence encoding the C terminal proline residue of the amino acid sequence of SEQ ID No. 29.

82. The method as claimed in claim 80 wherein the sequence comprises a second detection tag.

83. The method as claimed in claim 82 wherein the second detection tag is located upstream of the sequence encoding the N terminal proline residue of the amino acid sequence of SEQ ID No. 29.

84. The method as claimed in claim 82 wherein the first and second detection tags are different.

85. A use of a sequence encoding the amino acid sequence comprising SEQ ID No. 29 for identifying a compound and/or a molecule for the prophylaxis and/or treatment of a virus.

86. The compound and/or molecule identified by the method as claimed in claim 70.

87. The method of treatment and/or prophylaxis of a viral disease such as Foot and Mouth disease comprising the step of administering an effective amount of a compound and/or a molecule as claimed in claim 86 to a mammal.

88. A kit comprising:
a translation system; and
a sequence encoding the amino acid sequence comprising SEQ ID No. 29.
